SEQUENCE LISTING (66 sequences)

SEQ ID NO: 1 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 2 - protein, 131 aa, Mus musculus
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKL

SEQ ID NO: 3 - protein, 10 aa, synthetic construct
GFTFSSYAMS

SEQ ID NO: 4 - protein, 17 aa, synthetic construct
AISGSGGSTYYADSVKG

SEQ ID NO: 5 - protein, 7 aa, synthetic construct
RYIAFDY

SEQ ID NO: 6 - protein, 11 aa, synthetic construct
RASQSISSYLN

SEQ ID NO: 7 - protein, 7 aa, synthetic construct
AASSLQS

SEQ ID NO: 8 - protein, 9 aa, synthetic construct
QQGGMPPDT

SEQ ID NO: 9 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSS

SEQ ID NO: 10 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 11 - protein, 237 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGGGSGGGGSGGGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 12 - protein, 14 aa, synthetic construct
GGGGSGGGGSGGGG

SEQ ID NO: 13 - protein, 446 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 358 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGGGSGGGGSGGGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 16 - protein, 6 aa, synthetic construct
SGGSGG

SEQ ID NO: 17 - protein, 446 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 228 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGSGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 20 - protein, 472 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGSGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKDIGGGSGGGGSGGGGEEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGSGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 21 - protein, 5 aa, synthetic construct
GGSGG

SEQ ID NO: 22 - protein, 443 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 482 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKGGGGSGGGGSGGGGSEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 24 - protein, 487 aa, synthetic construct
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 25 - protein, 238 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 26 - protein, 15 aa, synthetic construct
GGGGSGGGGSGGGGS

SEQ ID NO: 27 - protein, 466 aa, synthetic construct
MGWSCIILFLVATATGVHSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 233 aa, synthetic construct
MGWSCIILFLVATATGVHSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 15 aa, synthetic construct
NKILVKQSPMLVAYD

SEQ ID NO: 30 - protein, 15 aa, synthetic construct
VKQSPMLVAYDNAVN

SEQ ID NO: 31 - protein, 15 aa, synthetic construct
PMLVAYDNAVNLSCK

SEQ ID NO: 32 - protein, 15 aa, synthetic construct
AYDNAVNLSCKYSYN

SEQ ID NO: 33 - protein, 15 aa, synthetic construct
AVNLSCKYSYNLFSR

SEQ ID NO: 34 - protein, 15 aa, synthetic construct
SCKYSYNLFSREFRA

SEQ ID NO: 35 - protein, 15 aa, synthetic construct
SYNLFSREFRASLHK

SEQ ID NO: 36 - protein, 15 aa, synthetic construct
FSREFRASLHKGLDS

SEQ ID NO: 37 - protein, 15 aa, synthetic construct
FRASLHKGLDSAVEV

SEQ ID NO: 38 - protein, 15 aa, synthetic construct
LHKGLDSAVEVCVVY

SEQ ID NO: 39 - protein, 15 aa, synthetic construct
LDSAVEVCVVYGNYS

SEQ ID NO: 40 - protein, 15 aa, synthetic construct
VEVCVVYGNYSQQLQ

SEQ ID NO: 41 - protein, 15 aa, synthetic construct
VVYGNYSQQLQVYSK

SEQ ID NO: 42 - protein, 15 aa, synthetic construct
NYSQQLQVYSKTGFN

SEQ ID NO: 43 - protein, 15 aa, synthetic construct
QLQVYSKTGFNCDGK

SEQ ID NO: 44 - protein, 15 aa, synthetic construct
YSKTGFNCDGKLGNE

SEQ ID NO: 45 - protein, 15 aa, synthetic construct
GFNCDGKLGNESVTF

SEQ ID NO: 46 - protein, 15 aa, synthetic construct
DGKLGNESVTFYLQN

SEQ ID NO: 47 - protein, 15 aa, synthetic construct
GNESVTFYLQNLYVN

SEQ ID NO: 48 - protein, 15 aa, synthetic construct
VTFYLQNLYVNQTDI

SEQ ID NO: 49 - protein, 15 aa, synthetic construct
LQNLYVNQTDIYFCK

SEQ ID NO: 50 - protein, 15 aa, synthetic construct
YVNQTDIYFCKIEVM

SEQ ID NO: 51 - protein, 15 aa, synthetic construct
TDIYFCKIEVMYPPP

SEQ ID NO: 52 - protein, 15 aa, synthetic construct
FCKIEVMYPPPYLDN

SEQ ID NO: 53 - protein, 15 aa, synthetic construct
EVMYPPPYLDNEKSN

SEQ ID NO: 54 - protein, 15 aa, synthetic construct
PPPYLDNEKSNGTII

SEQ ID NO: 55 - protein, 15 aa, synthetic construct
LDNEKSNGTIIHVKG

SEQ ID NO: 56 - protein, 15 aa, synthetic construct
KSNGTIIHVKGKHLC

SEQ ID NO: 57 - protein, 15 aa, synthetic construct
TIIHVKGKHLCPSPL

SEQ ID NO: 58 - protein, 15 aa, synthetic construct
VKGKHLCPSPLFPGP

SEQ ID NO: 59 - protein, 15 aa, synthetic construct
KHLCPSPLFPGPSKP

SEQ ID NO: 60 - protein, 493 aa, synthetic construct
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 61 - protein, 683 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 62 - protein, 215 aa, synthetic construct
EIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 684 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGSEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 64 - protein, 214 aa, synthetic construct; identical to SEQ ID NO: 14

SEQ ID NO: 65 - protein, 471 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGSGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKDIGGGSGGGGSGGGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSGGSGGDIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIK

SEQ ID NO: 66 - protein, 395 aa, synthetic construct
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPGSGLNDIFEAQKIEWHEGSHHHHHHGSDDDDKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0368738 A1
(43) Pub. Date: Dec. 4, 2025

(54) ANTI-CD28 ANTIBODIES

(71) Applicant: PHILOGEN S.P.A, SIENA (IT)

(72) Inventors: Roberto DE LUCA, Otelfingen (CH); Abdullah ELSAYED, Otelfingen (CH); Frederik PEISSERT, Otelfingen (CH); Louis PLUSS, Otelfingen (CH); Dario NERI, Siena (IT)

(21) Appl. No.: 18/877,930

(22) PCT Filed: Jul. 21, 2023
(86) PCT No.: PCT/EP2023/070320

§ 371 Date: Dec. 20, 2024

(30) Foreign Application Priority Data
Jul. 22, 2022 (EP) 22186570.2
May 23, 2023 (EP) 23174908.6

(51) Int. Cl. C07K 16/28; C07K 16/30

(52) U.S. Cl. CPC C07K 16/2818; C07K 16/2809; C07K 16/3007; C07K 2317/31; C07K 2317/33; C07K 2317/34; C07K 2317/622; C07K 2317/75

(57) ABSTRACT

The application relates to the diagnosis and treatment of diseases, including cancer, chronic infectious diseases, autoimmune diseases, inflammatory disorders, as well as the prevention of transplant rejection. The invention provides, and involves the use of, antibody molecules that bind CD28 in a non super-agonistic manner and which also bind to CTLA-4. The antibody molecules may form part of a bispecific molecule which binds e.g. a tumor associated antigen or a further T cell antigen, such as CD3.

This application claims priority from European Patent Application No. 22186570.2 filed on 22 Jul. 2022 and from European Patent Application No. 23174908.6 filed on 23 May 2023, the contents and elements of which are herein incorporated by reference for all purposes.

### FIELD OF THE INVENTION

The present invention relates to the diagnosis and treatment of diseases, including cancer, chronic infectious diseases, autoimmune diseases, inflammatory disorders as well as in the prevention of transplant rejection. The invention provides, and involves the use of, antibody molecules that bind CD28 from humans and mouse in a non super-agonistic manner and which also bind to CTLA-4. The antibody molecules may form part of a bispecific molecule which additionally binds a tumor associated antigen or a further T cell antigen.

### BACKGROUND

CD28 is a homodimeric glycoprotein molecule that is constitutively expressed on the surface of most T cells (around 95% of CD4+ and 50% of CD8+ T cells; Damle et al., 1983). The main function of CD28 is to provide a crucial co-stimulatory signal that enhances T cell proliferation, survival, and production of key cytokines such as interleukin 2, IFN-gamma, and TNF-alpha, as outlined below (Jenkins et al., 1991; June et al., 1987; Martin et al., 1986; Weiss et al., 1986).

T cell activation is defined using a two-signal model (Mueller et al., 1989; Jenkins et al., 1991; Bretscher and Cohn, 1970). Signal one is regulated by T cell receptor (TCR)/CD3 complex engagement (Allison et al., 1982; Hedrick et al., 1984; Yanagi et al., 1984). TCR engagement is induced through recognition of antigenic peptides presented by major histocompatibility complex (pMHC) on antigen presenting cells (APCs), such as B cells (La Gruta et al., 2018; Davis and Bjorkman, 1988). TCR itself lacks intracellular signalling domains and therefore the association with CD3 and other co-receptors (e.g. CD4 and CD8) is essential for the generation of an activation signal (Weiss and Stobo 1984; Ohashi et al., 1985; Weiss et al., 1986; Janeway, 1988). However, the TCR/CD3 complex alone is not sufficient for full T cell activation. In fact, the absence of an additional signal induces T cell exhaustion and thus impaired activation of T cells (Mueller et al., 1989).

Signal two is directed by the engagement of co-stimulatory receptors found on the surface membrane of T cells with counter receptors on APCs (Mueller et al., 1989; Jenkins et al., 1991; Bretscher and Cohn, 1970). CD28 is an example of such a co-stimulatory receptor and binds to its counter receptors CD80 and CD86 on APCs in order to provide co-stimulation (Freeman et al., 1989; Freeman et al., 1993 (a); Freeman et al., 1993 (b); Azuma et al., 1993; Caux et al., 1994).

In addition to CD28, CTLA-4 is a second ligand for CD80/CD86. CTLA-4 is upregulated in CD8+ T cells after activation and functions as immune checkpoint by providing an inhibitory signal (Brunet et al., 1987). The use of monoclonal antibodies (mAbs) to block CTLA-4, thereby reducing this inhibitory signal, has revolutionized the field of cancer immunotherapy (Krummel and Allison, 1995; Leach et al., 1996; Kwon et al., 1997). CTLA-4 has been shown to share significant structural homology with CD28 and outcompetes CD28 for binding to CD80/CD86 with an at least 20-fold higher affinity (Linsley et al., 1991; Peach et al., 1994). The CD28/CTLA-4 pathway is the prototypic co-signalling pathway in T-cells, with CTLA-4 co-inhibition acting as the counter signal to CD28 co-stimulation, as they bind the same receptors (CD80 and CD86). Since CD28 co-stimulation is crucial for T-cell activation, immunomodulation via blockade of this pathway using an antagonist anti-CD28 antibody is a promising approach to prevent inappropriate T-cell activation in the setting of transplantation and also to potentially treat T-cell mediated autoimmune diseases (Crepeau and Ford 2017).

Due to its role in the activation of T cells, CD28 has been identified as a therapeutic target, for example in the treatment of cancer. A number of antibodies which bind human CD28 have been described in the art. These can be broadly classified as either conventional (agonistic) anti-CD28 antibodies, or non-conventional (super-agonistic) anti-CD28 antibodies (Tacke et al., 1997; Lühder et al., 2003).

Combination of agonistic antibodies against both the TCR/CD3 complex and CD28 are sufficient to fully activate T cells through cross-linking and therefore can replace MHC and CD80/86 signalling, respectively. Conventional anti-CD28 mAbs bind close to the natural binding site of CD80/CD86 and provide co-stimulation only in the presence of TCR/CD3 signalling (Lühder et al., 2003).

Conversely, super-agonistic anti-CD28 mAbs bind to the laterally exposed C″D loop of CD28 and can fully activate T cells without the need for TCR/CD3 complex engagement (Lühder et al., 2003; Tacke et al., 1997). In rat models, this unusual class of antibodies induces potent proliferation of T cells without clear toxicity (Tacke et al., 1997; Rodríguez-Palmero et al., 1999). At low doses, only regulatory T cells (Tregs) were activated, while at high doses both Tregs and conventional T cells were expanded and therefore preferential activation of T cells at different doses was hypothesized (Lin et al., 2003; Beyersdorf et al., 2005).

As a result, a humanized anti-CD28 antibody in IgG4 format (TGN1412) that binds to both human and cynomolgus monkey CD28 was developed for different therapeutic indications (Lühder et al., 2003). In cynomolgus monkeys, TGN1412 was well tolerated up to a dose of 50 mg/kg and therefore an initial dose of 0.1 mg/kg was proposed to be safe for human treatment (Pallardy and Hünig, 2010; Hanke 2006). However, in 2006, a Phase I clinical trial was conducted in which all six volunteers receiving TGN1412 developed a life-threatening cytokine release syndrome (CRS) with multiple-organ failures, resulting in termination of the clinical trial (Suntharalingam et al., 2006).

Many factors contributed to the failure to predict the toxicity of TGN1412 in the preclinical phase. Firstly, adding TGN1412 to human PBMCs in vitro does not induce cytokine release, unless the cells are artificially immobilized on cell culture wells (Stebbings et al., 2007). Secondly, a subset of T cells (known as tissue resident CD4 effector memory; CD4EM) are the main source of CRS. Toxicity could therefore not be detected in the young and clean laboratory rats that lack such cells. The cytokine release in rodents is also quenched by Treg cells, which are fuelled by IL-2 produced by conventional T cells (Römer et al., 2011; Eastwood et al., 2010). Finally, and most importantly, CD4EM cells in cynomolgus monkeys were found to downregulate CD28 and therefore toxicity in humans could not be predicted from testing in these non-human primates (Eastwood et al., 2010).

No anti-CD28 antibodies are currently approved for cancer therapy in human patients. Thus, there remains a need in the art for further anti-CD28 antibodies which have a more favourable toxicity profile than TGN1412, e.g. for use in the treatment of cancer.

The present invention has been devised in light of the above considerations.

### SUMMARY OF THE INVENTION

The present inventors have developed human antibody molecules which bind human CD28 and provide a strong co-stimulatory signal, but do not activate T cells without TCR/CD3 engagement (i.e. these antibodies are not super-agonistic). This is in contrast to the known anti-CD28 antibody TGN1412 for which T cell activation and proliferation was observed in the absence of TCR/CD3 engagement (Example 4). Preferably, the antibodies of the present invention are agonistic anti-CD28 antibodies.

The strong co-stimulatory signal of the antibodies of the present invention and the absence of super-agonistic activity is expected to translate into a more favourable toxicity profile than that observed with TGN1412, while boosting the immune system to fight cancer or chronic infectious diseases. Advantageously, the co-stimulatory signal provided by the antibodies of the present invention is stronger than that provided by TGN1412 (Example 3).

Furthermore, the antibodies of the present invention also bind to CTLA-4 (Example 5). CTLA-4 and CD28 both share a conserved binding motif (MYPPPY) that is essential for binding to CD80/CD86 (Peach et al., 1994). Unlike TGN1412, the antibodies of the present invention bind to an epitope of CD28 which contains the MYPPY binding motif (Example 6).

Cross-reactivity with CTLA-4 means that the antibodies of the invention have the potential to act as a checkpoint inhibitor, further boosting the anti-cancer immune response, in addition to their co-stimulatory activity. The property of cross-reactivity with CTLA-4 is believed not to be an inherent feature of all non-super agonistic anti-CD28 antibodies, for example such a property has not been shown for the anti-CD28 antibodies disclosed in U.S. Pat. No. 20,190,389951A1.

The antibodies of the present invention are also cross-reactive with murine CD28. Cross-reactivity with murine CD28 provides avenues for evaluating efficacy of the anti-CD28 antibody molecules. Due to the anatomical, physiological, and genetic similarity to humans, the mouse represents a useful animal model for the evaluation of anti-CD28 antibodies, as well as other therapeutics. Advantages of mice include their small size, ease of maintenance, short life cycle, and abundant genetic resources, meaning that mice provide a promising animal model for translational studies to determine the efficacy of anti-CD28 therapeutics.

In a first aspect, the present invention thus relates to antibody molecules that bind CD28. The antibody molecule preferably comprises the HCDR1, HCDR2, and HCDR3 sequences of the “AE2P” antibody set forth in SEQ ID NOs 3, 4 and 5, respectively, and/or the LCDR1, LCDR2 and LCDR3 sequences of the AE2P antibody set forth in SEQ ID NOs 6, 7 and 8, respectively. In a preferred embodiment, the antibody molecule comprises the VH domain or VL domain sequence, but preferably the VH domain and VL domain sequence, of the AE2P antibody molecule set forth in SEQ ID NOs 9 and 10, respectively. The VH and VL domains of the antibody molecule may be linked by a linker, such as the linker set forth in SEQ ID NO: 12.

The antibody molecules of the present invention preferably bind human CD28, as well as CD28 from mice (Mus musculus), referred to as “murine” CD28 herein. The sequence of the human extracellular domain of CD28 is shown in SEQ ID NO: 1, while the sequence of the mouse extracellular domain of CD28 is shown in SEQ ID NO: 2.

The antibody molecules of the present invention are preferably human or humanised antibodies. Most preferably, the antibodies of the present invention are fully human antibodies. Fully human antibodies are advantageous due to their lower potential for immunogenicity.

An antibody molecule, as referred to herein, may be in any suitable format. In some embodiments, the antibody may bind CD28 monovalently. In some embodiments, the antibody may bind CD28 bivalently. Many antibody molecule formats are known in the art and include both complete antibody molecule molecules, such as IgG, as well as antibody molecule fragments, such as a single chain Fv (scFv) or single chain diabody (scDb). The term “antibody molecule” as used herein encompasses both complete antibody molecule molecules and fragments of antibody molecules, in particular antigen-binding fragments. Preferably, an antibody molecule comprises a VH domain and a VL domain. In a preferred embodiment, the antibody molecule is or comprises a scFv, is a small immunoprotein (SIP), is a diabody (Db), is a single-chain diabody (scDb), or is a (complete) IgG molecule, such as an IgG1, IgG2a, or IgG4 molecule. The sequence of the AE2P antibody in single chain Fv (scFv) format is shown in SEQ ID NOs: 11 and 25. The sequence of the AE2P antibody in diabody (Db) format is shown in SEQ ID NO: 19. The sequence of the AE2P antibody in single chain diabody (scDb) format is shown in SEQ ID NO 65. Alternatively, the AE2P antibody in single chain diabody (scDb) format may have the sequence set forth in SEQ ID NO: 20. The sequence of the AE2P antibody in small immunoprotein (SIP) format is shown in SEQ ID NO: 15. The sequence of the AE2P light chain is shown in SEQ ID NO: 14, while the sequence of the AE2P heavy chain in IgG₁ format is shown in SEQ ID NO: 13, the sequence of the AE2P heavy chain in IgG_{2A} format is shown in SEQ ID NO: 17, and the sequence of the AE2P heavy chain in IgG₄ format is shown in SEQ ID NO: 22.

An antibody molecule of the present invention may be used as is, or may be conjugated to a molecule to provide a conjugate. The choice of molecule conjugated to the antibody molecule will depend on the intended application of the conjugate. For example, where the conjugate is intended for the treatment of a disease or disorder, the conjugate may comprise an antibody molecule of the invention and a bioactive agent. The bioactive agent may be a pro-inflammatory agent. Where the conjugate is intended for use in imaging, detecting, or diagnosing a disease or disorder, the conjugate may comprise an antibody molecule of the invention and a detectable label or marker molecule, such as a radioisotope, e.g. a non-therapeutic radioisotope. Depending on the molecule conjugated to the antibody molecule, the conjugate may be or may comprise a single-chain protein. Where the conjugate is a single-chain protein, the entire protein can be expressed as a single polypeptide or fusion protein. In this case, the molecule may be conjugated to the antibody molecule by means of a peptide linker. Fusion proteins have the advantage of being easier to produce and purify since they consist of a single species. This facilitates production of clinical-grade material. Alternatively, the molecule may be conjugated to the antibody molecule by means of a cleavable linker.

An antibody molecule of the present invention may form part of a bispecific binding molecule. In some embodiments, the bispecific binding molecule comprises an antigen-binding site which binds a tumor associated antigen, e.g. fibroblast activation protein (FAP), a splice isoform of fibronectin such as the ED-A, ED-B or IIICS isoforms of fibronectin, carbonic anhydrase IX (CAIX), carcinoembryonic antigen (CEA), Mucin-16, prostate-specific membrane antigen (PSMA), or splice isoforms of tenascin-C such as the A1, A2, B, C or D isoform of tenascin-C. In some embodiments, the bispecific binding molecule comprises an antigen-binding site which binds a second T cell antigen, e.g. CD3. Bispecific binding molecules as used according to the present invention include bispecific antibodies, and may be selected from IgG-appended antibodies with an additional antigen-binding moiety (e.g. IgG-(scFv)₂ and IgG-(scFv)) or small recombinant bispecific antibody formats (e.g. bispecific T-cell engager (BiTE™) and scDb-scFv) or any other molecular format which includes a binding molecule specific for a given target conjugated to one or two different binding molecules specific for one or two different targets. Examples of bispecific binding molecules can be found in Kontermann 2012 (page 186 FIG. 2) the content of which is incorporated herein by reference. The sequence of the AE2P antibody and the anti-FAP antibody 7NP2 in bispecific T cell engager (BiTE™) format is shown in SEQ ID NO: 23. The heavy and light chain sequences of an anti-human CEA antibody and the AE2P antibody in IgG-(scFv)₂ format, wherein the anti-human CEA antibody is in IgG format and the AE2P antibody is in scFv format (2+2; format 1, FIG. 13), are shown in SEQ ID NOs 61 and 62, respectively. The heavy and light chain sequences of the AE2P antibody and an anti-human CEA antibody in IgG-(scFv)₂ format, wherein the AE2P antibody is in IgG format and the anti-human CEA antibody is in scFv format (2+2; format 2, FIG. 13), are shown in SEQ ID NOs 63 and 64, respectively.

The invention also provides isolated nucleic acids encoding the antibody molecules and conjugates of the invention. The skilled person would have no difficulty in preparing such nucleic acids using methods well-known in the art. An isolated nucleic acid may be used to express the antibody molecule or conjugate of the invention, for example by expression in a bacterial, yeast, insect or mammalian host cell. Preferred host cells are E. coli and CHO-S cells. The nucleic acid will generally be provided in the form of a recombinant expression vector for expression. Host cells in vitro comprising such nucleic acids and expression vectors are part of the present invention, as is their use for expressing the antibody molecules and conjugates of the invention, which may subsequently be purified from cell culture and optionally formulated into a pharmaceutical composition.

An antibody molecule or conjugate of the invention may be provided for example in a pharmaceutical composition, and may be employed for medical use as described herein, either alone or in combination with one or more further therapeutic agents. For example, an antibody molecule or conjugate of the invention may be employed for a medical use as described herein in combination with a CD3 agonist, for example an antibody which binds human CD3. Exemplary further therapeutic agents that may be combined with, or administered in association with, an antibody molecule of the present invention include, e.g., chemotherapy (e.g., anti-cancer chemotherapy, for example, paclitaxel, docetaxel, vincristine, cisplatin, carboplatin or oxaliplatin), radiation therapy, a checkpoint inhibitor, e.g a checkpoint inhibitor that targets PD-1 (e.g., an anti-PD-1 antibody such as pembrolizumab, nivolumab, or cemiplimab (see U.S. Pat. No. 9,987,500)), CTLA-4, LAG3, or TIM3, a costimulatory agonist antibody that targets e.g. GITR, OX40, or 4-1 BB, or a second anti-CD28 antibody, such as a second costimulatory CD28 bispecific antibody.

Alternatively, the antibody molecule or conjugate of the invention may be provided in a diagnostic composition and may be employed for diagnostic use as described herein.

The present invention also relates to an antibody molecule or conjugate of the invention for use in a method for treatment of the human or animal body by therapy. For example, an antibody molecule or conjugate of the invention may be for use in a method of treating cancer, a chronic infectious disease, an autoimmune disease, and/or an inflammatory disorder in a patient, or for use in a method for preventing transplant rejection in a patient.

The invention also relates to a method of treating cancer, a chronic infectious disease, an autoimmune disease, and/or an inflammatory disorder in a patient, or a method of preventing transplant rejection, the method comprising administering a therapeutically effective amount of an antibody molecule or conjugate of the invention to the patient. The use of an antibody molecule or conjugate of the invention for the manufacture of a medicament for the treatment of cancer, a chronic infectious disease, an autoimmune disease, and/or an inflammatory disorder in a patient, or the manufacture of a medicament for the prevention of transplant rejection, is also contemplated. Examples of chronic infectious diseases, as referred to herein, include chronic hepatitis B infection (HBV), human immunodeficiency virus (HIV) infection, and tuberculosis. Examples of autoimmune diseases which may be treated using an antibody molecule or conjugate of the invention herein include lupus erythematosus, rheumatoid arthritis, and psoriatic arthritis. An inflammatory or autoimmune disease which may treated using an antibody molecule or conjugate of the invention includes inflammatory bowel disease (IBD), such Crohn's disease or ulcerative colitis.

In a preferred embodiment, the antibody molecule or conjugate of the invention is for use in a method of treating cancer.

A further aspect of the invention relates to an antibody molecule or conjugate of the invention for use in a method of imaging, detecting, or diagnosing cancer, a chronic infectious disease, an autoimmune disease, and/or an inflammatory disorder in a patient. The invention further relates to a method of imaging, detecting, or diagnosing cancer, a chronic infectious disease an autoimmune disease, and/or an inflammatory disorder in a patient comprising administering an antibody molecule or conjugate of the invention to the patient. The method may be an in vitro or an in vivo method. Also encompassed within the scope of the invention is the use of an antibody molecule or conjugate of the invention for the manufacture of a diagnostic product for imaging, detecting, or diagnosing cancer, a chronic infectious disease, an autoimmune disease, and/or an inflammatory disorder.

In a preferred embodiment, the antibody molecule or conjugate of the invention is for use in a method of imaging, detecting, or diagnosing cancer.

A patient, as referred to herein, is preferably a human patient.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the biochemical characterization of AE2P in scFv format. FIG. 1A shows the results of size exclusion chromatogram of scFv (AE2P). The monomeric form of the scFv was eluted from the S75i GL column at 11.5 mL as expected. FIG. 1B shows the results of SDS-PAGE analysis of scFv (AE2P). The scFv had the expected size of 25 kDa under non-reducing (NR) and reducing (R) conditions, respectively. FIG. 1C shows titration ELISA of scFv (AE2P) on human CD28-Fc fusion protein. The data shows an approximate EC₅₀ of 31 nM confirming the binding of AE2P to CD28 .

FIG. 2 shows the biochemical characterization of AE2P in IgG format. FIG. 2A shows the results of size exclusion chromatogram of IgG4 (AE2P). The IgG₄ was eluted from the S200i 10/300 GL column at 11.9 mL as expected. FIG. 2B shows the results of SDS-PAGE analysis of IgG₄ (AE2P). The IgG₄ had the expected size of 150 kDa under non-reducing conditions and 25 and 50 kDa under reducing condition. FIG. 2C shows flow cytometry analysis of AE2P IgG₄ and TGN1412 IgG₄ (also named TGN) binding to primary human T cells. Dotted lines represent the isotype negative control, while solid filled represent the shift in fluorescence upon binding. This confirms binding of both AE2P IgG₄ and TGN1412 IgG₄ to primary human T cells expressing CD28.

FIG. 3 shows flow cytometric analysis of AE2P IgG₄ on various negative cell lines. Dotted lines represent the isotype negative control, while solid line represent AE2P IgG₄. This confirms the specific binding of AE2P IgG₄ to primary human T cells and not to other negative cell lines that do not express CD28.

FIG. 4 shows the proliferation of human PBMCs after 3 days of incubation with either OKT3 alone or in combination with AE2P IgG₄, TGN1412 IgG₄ (also named TGN) or isotype IgG₄. This confirms the co-stimulatory effect on proliferation of human PBMCs using anti-CD28 antibodies (AE2P IgG₄ and TGN1412 IgG₄) in combination with anti-CD3 (OKT3). The data also demonstrates that AE2P IgG₄ provides a greater co-stimulatory effect on the proliferation of human PBMCs compared with TGN1412 IgG₄.

FIG. 5 shows the cytokine release from human PBMCs after 3 days of incubation with either OKT3 alone or in combination with AE2P IgG₄, TGN1412 IgG₄ (also named TGN) or isotype IgG₄. FIG. 5A shows the cytokine release of human IL-2. This confirms the increase in IL-2 secretion from human PBMCs when using anti-CD28 antibodies (AE2P IgG₄ and TGN1412 IgG₄) in combination with anti-CD3 (OKT3). FIG. 5B shows the cytokine release of human IFN-gamma. This confirms the increase in IFN-gamma secretion from human PBMCs when using anti-CD28 antibodies (AE2P IgG₄ and TGN1412 IgG₄) in combination with anti-CD3 (OKT3). FIG. 5C shows the cytokine release of human TNF-alpha. This confirms the increase in TNF-alpha secretion from human PBMCs when using anti-CD28 antibodies (AE2P IgG₄ and TGN1412 IgG₄) in combination with anti-CD3 (OKT3). The data also demonstrates that AE2P IgG₄ provides a greater co-stimulatory effect on the release of all three cytokines compared with TGN1412 IgG₄.

FIG. 6 shows the proliferation of human PBMCs after incubation with either AE2P IgG₄, TGN1412 IgG₄ (also named TGN) or isotype IgG₄ in the absence of OKT3. FIG. 6A shows the proliferation of human PBMCs after 3 days. This confirms the lack of proliferation of human PBMCS after 3 days of incubation with AE2P IgG₄ alone, unlike TGN1412 IgG₄. FIG. 6B shows the proliferation of human PBMCs after 5 days. This confirms the lack of proliferation of human PBMCS after 5 days of incubation with AE2P IgG₄ alone, unlike TGN1412 IgG₄.

FIG. 7 shows the cytokine release from human PBMCs after incubation with either AE2P IgG₄, TGN1412 IgG₄ (also named TGN) or isotype IgG₄ in the absence of OKT3. FIG. 7A shows the cytokine release of human IL-2. This confirms the lack of IL-2 secretion from human PBMCS after 3 days of incubation with AE2P IgG₄ alone, unlike TGN1412 IgG₄. FIG. 7B shows the cytokine release of human IFN-gamma. This confirms the lack of IFN-gamma secretion from human PBMCS after 3 days of incubation with AE2P IgG₄ alone, unlike TGN1412 IgG₄. FIG. 7C shows the cytokine release of human TNF-alpha. This confirms the lack of TNF-alpha secretion from human PBMCS after 3 days of incubation with AE2P IgG₄ alone, unlike TGN1412 IgG₄.

FIG. 8 shows the upregulation of activation markers on human PBMCs after 3 days of incubation with either AE2P IgG₄, TGN1412 IgG₄ (also named TGN) or isotype IgG₄ in the absence of OKT3. FIG. 8A shows the upregulation of CD69 on CD4 positive T cells. This confirms the lack in upregulation of CD69 on CD4 positive T cells in human PBMCS after 3 days of incubation with AE2P IgG₄ alone, unlike TGN1412 IgG₄. FIG. 8B shows the upregulation of CD69 on CD8 positive T cells. This confirms the lack in upregulation of CD69 on CD8 positive T cells in human PBMCS after 3 days of incubation with AE2P IgG₄ alone, unlike TGN1412 IgG₄. FIG. 8C shows the upregulation of CD25 on CD4 positive T cells. This confirms the lack in upregulation of CD25 on CD4 positive T cells in human PBMCS after 3 days of incubation with AE2P IgG₄ alone, unlike TGN1412 IgG₄. FIG. 8D shows the upregulation of CD25 on CD8 positive T cells. This confirms the lack in upregulation of CD25 on CD8 positive T cells in human PBMCS after 3 days of incubation with AE2P IgG₄ alone, unlike TGN1412 IgG₄.

FIG. 9 shows the binding of AE2P to human and mouse CD28 Fc. FIG. 9A shows the binding of AE2P IgG₄ to human CD28 Fc by titration ELISA versus binding to non-relative protein. FIG. 9B shows the binding of AE2P IgG₄ to murine CD28 Fc by titration ELISA versus binding to non-relative protein. FIG. 9C shows the binding of TGN1412 (also named TGN) to human CD28 Fc by titration ELISA versus binding to non-relative protein. FIG. 9D shows the lack of binding of TGN1412 (also named TGN) to murine CD28 Fc by titration ELISA versus binding to non-relative protein. FIG. 9E shows the binding of AE2P or TGN1412 to primary mouse T cells, as determined by flow cytometry. Dotted lines represent the isotype negative control, while solid line represent AE2P or TGN1412. The results show that AE2P IgG₄, but not TGN1412, binds to primary mouse T cells.

FIG. 10 shows the binding of AE2P or TGN1412 (also named TGN) to human CTLA-4 by titration ELISA. This confirms the binding of AE2P IgG₄, but not TGN1412 IgG₄, to human CTLA-4.

FIG. 11 shows the epitope recognized by AE2P. FIG. 11A shows the membrane scan after incubation with AE2P IgG₄. FIG. 11B shows the peptide array sequence of CD28 used for PepSpot. Dark grey highlights the dark spot region on the membrane (array 24-25), while light grey highlights the light spot region on the membrane (array 11-13). FIG. 11C and FIG. 11D show the alignment of the epitope mapping on the CD28 crystal structure in a cartoon representation (FIG. 11C) and a surface representation (FIG. 11D).

FIG. 12 shows in vitro cell lysis of WI-38 cell line using the bispecific T-cell engager “L19-OKT3” alone or “L19-OKT3” in combination with a further bispecific T-cell engager “7NP2-AE2P”. The combination produces increased cell lysis of the WI-38 cell line, indicating a potent and specific synergism between AE2P in a bispecific format and CD3-engaging bispecific antibodies.

FIG. 13 shows examples of some molecular formats in which AE2P can be used for cancer immunotherapy and/or for use in treating chronic infectious diseases, autoimmune diseases, inflammatory disorders and/or preventing transplant rejection.

FIG. 14 shows the results of an in vitro super-agonism assay on mouse T cells. Data are presented as mean ±SD from technical triplicates. FIG. 14A shows the proliferation of mouse T cells after incubation with either AE2P IgG₄, TGN1412 IgG₄ (also named TGN) or no antibody in the absence of the anti-mouse CD3 antibody 2C11. This confirms the lack of proliferation of mouse T cells after 5 days of incubation with AE2P IgG₄ alone. FIG. 14B shows the quantification of cytokines (IL-2 and IFN-γ) by ELISA after 3 days incubation with either AE2P IgG₄, TGN1412 IgG₄ or no antibody in the absence of anti-mouse CD3 antibody 2C11. This confirms the lack of IL-2 and IFNγ secretion from mouse T cells after 3 days incubation with AE2P IgG₄ alone. FIG. 14C shows the expression of an early activation marker (CD69) and a late activation marker (CD25) by mouse CD3+ T cells after 3 days of incubation with either AE2P IgG₄, TGN1412 IgG₄, or no antibody in the absence of anti-mouse CD3 antibody 2C11. This confirms the lack in upregulation of both CD69 and CD25 in mouse T cells after 3 days of incubation with AE2P IgG₄ alone.

FIG. 15 shows the results of an in vitro co-stimulation assay on mouse T cells. FIG. 15A shows the proliferation of mouse T cells after 3 days of incubation with anti-mouse CD3 antibody 2C11 alone, or in combination with AE2P IgG₄, or TGN1412 IgG₄ (also named TGN). This confirms the co-stimulatory effect of AE2P IgG₄, but not TGN1412 IgG₄, on the proliferation of mouse T cells in the presence of antibody 2C11. FIG. 15B shows the release of cytokines (IL-2 and IFN-γ) from mouse T cells after 3 days of incubation with either 2C11 alone, or in combination with AE2P IgG₄ or TGN1412 IgG₄. This confirms the co-stimulatory effect of AE2P IgG₄, but not TGN1412 IgG₄, on the secretion of IL-2 by mouse T cells in combination with 2C11 (left panel). No change was observed in IFN-γ release with either AE2P IgG₄ or TGN1412 IgG₄ (right panel).

FIG. 16 shows the biochemical characterization of an anti-CD3/anti-ED-B bispecific T-cell engager (BiTE™) and the results of an in vitro co-stimulation and cell killing assay with the anti-CD3/anti-ED-B BiTE™ molecule. Data are presented as mean±SD (n=3 from technical replicates). FIG. 16A shows the results of size exclusion chromatogram of the anti-CD3/anti-ED-B BITE™. The anti-CD3/anti-ED-B BiTE™ was eluted from column as expected. FIG. 16B shows the results of SDS-PAGE analysis of the anti-CD3/anti-ED-B BiTE™. The anti-CD3/anti-ED-B BITE™ had the expected size under non-reducing (NR) and reducing (R) conditions, respectively. FIG. 16C shows the binding of the anti-CD3/anti-ED-B BiTE™ to the effector human T-cells (left panel) and on the target human cells WI-38 (right panel) as analysed by flow cytometry. The grey area corresponds to the fluorescence intensity from anti-CD3/anti-EDB BITE™ and the line to the fluorescence intensity from an isotype control. FIG. 16D shows target cell lysis of the ED-B⁺WI-38 target cell line using the anti-CD3/anti-EDB BiTE™ “PUB4-L19” alone, or “PUB4-L19” in combination with either TGN1412 IgG₄ (also named TGN) or AE2P IgG₄. A potent synergistic effect was observed when the BiTE™ “PUB4-L19” was combined with AE2P IgG₄, as reflected by the approximately twofold increase in cell lysis seen relative to the PUB4-L19 BITE™ only. The stimulatory activity seen with AE2P IgG₄ was only observed at concentrations of the anti-CD3/anti-EDB BiTE™ (PUB4-L19) that were initially active when added as a single agent (i.e., at 1 and 10 nM), unlike the superagonistic TGN1412 (also named TGN) IgG₄ antibody, which showed activity in the absence of CD3 co-stimulation (i.e., also at 0 and 0.1 nM). Similar results were observed when assessing CD25 expression (FIG. 16E), IFN-γ release (FIG. 16F), and T-cell proliferation (FIG. 16G).

FIG. 17 shows the biochemical characterization of the anti-human CEA IgG/AE2P (scFv)₂ bispecific antibody (2+2; format 1, FIG. 13). FIG. 17A shows the results of size exclusion chromatogram of the anti-human CEA IgG/AE2P (scFv)₂ bispecific antibody. The bispecific antibody was eluted from the S200i 10/300 GL column as expected. FIG. 17B shows the results of SDS-PAGE analysis of the anti-human CEA IgG/AE2P (scFv)₂ bispecific antibody. The bispecific antibody had the expected size under non-reducing (NR) and reducing (R) conditions, respectively. FIG. 17C shows the binding of the anti-human CEA IgG/AE2P (scFv)₂ bispecific antibody on primary mouse T cells as analysed by flow cytometry. FIG. 17D shows the binding of the anti-human CEA IgG/AE2P (scFv)₂ bispecific antibody on target cells expressing human CEA (C51.CEA).

FIG. 18 shows the biochemical characterization of the AE2P IgG/anti-human CEA (scFv)₂ bispecific antibody (2+2; format 2, FIG. 13). FIG. 18A shows the results of size exclusion chromatogram of the AE2P IgG/anti-human CEA (scFv)₂ bispecific antibody. The bispecific antibody was eluted from the S200i 10/300 GL column as expected. FIG. 18B shows the results of SDS-PAGE analysis of the AE2P IgG/anti-human CEA (scFv)₂ bispecific antibody. The bispecific antibody had the expected size under non-reducing (NR) and reducing (R) conditions, respectively. FIG. 18C shows the binding of the AE2P IgG/anti-human CEA (scFv)₂ bispecific antibody on primary mouse T cells as analysed by flow cytometry. FIG. 18D shows the binding of the AE2P IgG/anti-human CEA (scFv)₂ bispecific antibody on target cells expressing human CEA (C51.CEA).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Antibody Molecule

The present invention provides antibody molecules that bind CD28. The antibody molecule binds human CD28 and murine CD28, preferably the extracellular domain of human CD28 and murine CD28. The extracellular domain of human CD28 and murine CD28 may comprise or consist of the sequence set forth in SEQ ID NOs 1 and 2, respectively. The antibody molecule is preferably capable of binding to CD28 expressed on the surface of a cell, such as a T cell. Methods for determining binding an antigen, such as human or murine CD28, are known in the art and include ELISAs and flow cytometry, for example. The antibody molecule may bind to CD28 monovalently. The antibody molecule may bind to CD28 bivalently. The antibody molecule may be an agonist of CD28. The antibody molecule may be an antagonist of CD28.

The antibody molecule preferably binds CD28 specifically. The term “specific” is applicable where the antibody molecule is specific for particular epitopes, such as epitopes on CD28, that are carried by a number of antigens, in which case the antibody molecule will be able to bind to the various antigens carrying the epitope. In the case of the present invention, CTLA-4 carries the same “MYPPPY” binding motif present in the epitope of CD28 to which the antibody binds. Therefore, the antibody is specific for particular epitopes which are present on both CD28 and CTLA-4.

The antibody molecule, in scFv format, preferably binds human CD28 with high affinity. The antibody molecule may further bind to murine CD28. The antibody molecule, in scFv format, preferably has an EC₅₀ for human CD28 of less than 35 nM. The EC₅₀ of an antibody molecule to a cognate antigen, such as human CD28 can be determined by titration ELISA, e.g. as detailed in the examples.

The antibody molecule is preferably monoclonal. The antibody molecule may be human or humanised, but preferably is a human antibody molecule.

The antibody molecule may be isolated, in the sense of being free from contaminants, such as antibodies able to bind other polypeptides, and/or serum components.

The antibody molecule may be natural or partly or wholly synthetically produced. For example, the antibody molecule may be a recombinant antibody molecule.

The antibody molecule may be an immunoglobulin, or an antigen-binding fragment thereof. For example, the antibody molecule may be an IgG, IgA, IgE or IgM molecule, preferably an IgG molecule, such as an IgG₁, IgG₂, IgG₃ or IgG₄ molecule, more preferably an IgG₁, IgG_{2A}, or IgG₄ molecule, more preferably an IgG₄ molecule, or an antigen-binding fragment thereof such as a single chain Fv fragment (scFv), a diabody (Db), a single chain diabody (scDb), or a small immunoprotein (SIP).

A single chain Fv molecule (scFv), is a molecule wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883). Diabodies are multivalent or multispecific fragments constructed by gene fusion (WO2013/014149; WO94/13804; Holliger et al. (1993a), Proc. Natl. Acad. Sci. USA 90 6444-6448). Single chain diabodies are molecules wherein two sets of VH and VL domains are connected together in sequence on the same polypeptide chain (Konterman & Muller, 1999). ScFv or diabody molecules may be stabilized by the incorporation of disulphide bridges linking the VH and VL domains (Reiter et al. (1996), Nature Biotech, 14, 1239-1245). A single chain Fv (scFv) may be comprised within a small immunoprotein (SIP), e.g. as described in (Li et al., (1997), Protein Engineering, 10:731-736). A SIP may comprise an scFv molecule fused to the CH4 domain of the human IgE secretory isoform IgE-S2 (ε_{S2}-CH4; Batista et al., (1996), J. Exp. Med., 184:2197-205) forming a homo-dimeric mini-immunoglobulin antibody molecule.

In some embodiments, the antibody molecule may be an antigen-binding fragment comprising an antigen-binding site for CD28. An antigen binding site may be provided by one or more antibody variable domains (e.g. a so-called Fd antibody fragment consisting of a VH domain). Preferably, an antigen binding site comprises an antibody light chain variable region (VL) and an antibody heavy chain variable region (VH).

The antigen-binding site of an antibody molecule of the invention, such as an immunoglobulin or antigen-binding fragment thereof, binds CD28. The antigen-binding site may comprise three CDRs, such as the three light chain variable domain (VL) CDRs or three heavy chain variable domain (VH) CDRs, but preferably comprises six CDRs, three VL CDRs and three VH CDRs. The three VH domain CDRs of the antigen-binding site may be located within an immunoglobulin VH domain and the three VL domain CDRs may be located within an immunoglobulin VL domain. The antibody molecule may comprise one or two antigen-binding sites for CD28. Where the antibody molecule comprises two antigen-binding sites these are preferably identical. The antibody molecule thus may comprise one VH and one VL domain but preferably comprises two VH and two VL domains, i.e. two VH/VL domain pairs, as is the case in naturally-occurring immunoglobulin molecules, scFvs, diabodies and single-chain diabodies, for example.

The antigen-binding site of the antibody molecule preferably comprises the three VL domain CDRs and/or the three VH domain CDRs of antibody AE2P. The VH and VL domain sequences of this antibody are set forth in SEQ ID NOs 9 and 10, respectively, and the sequences of the CDRs of the AE2P antibody may be readily determined from these VH and VL domain sequences by the skilled person using routine techniques. The CDR sequences may, for example, be determined according to Kabat et al., “Sequences of Proteins of Immunological Interest,” National Institutes of Health, Bethesda, Md. (1991). In a preferred embodiment, the antigen-binding site of the antibody molecule comprises the HCDR1, HCDR2, and HCDR3sequences set forth in SEQ ID NOs 3, 4 and 5, respectively, and the LCDR1, LCDR2 and LCDR3 sequences set forth in SEQ ID NOs 6, 7 and 8, respectively.

In a further preferred embodiment, the antigen-binding site may comprise the VH domain (SEQ ID NO: 9) and/or VL domain (SEQ ID NO: 10) of antibody AE2P, but preferably comprises the VH domain and VL domain of antibody AE2P.

The antibody molecule may also comprise a variant of a CDR, VH domain, VL domain, heavy chain or light chain sequence, as described herein. Suitable variants can be obtained by means of methods of sequence alteration, or mutation, and screening. In a preferred embodiment, an antibody molecule comprising one or more such variant sequences retain one or more of the functional characteristics of the parent antibody molecule, such as binding specificity and/or binding affinity for human, or murine CD28.

The antibody molecule may comprise a VH domain which has at least 70%, more preferably at least 75%, at least 80%, at least 85%, at least 90%, at least 95%, at least 96%, at least 97%, at least 98%, at least 99% or 100%, sequence identity to the VH domain of antibody AE2P (SEQ ID NO: 9).

The antibody molecule may comprise a VL domain with at least 70%, more preferably one of at least 75%, at least 80%, at least 85%, at least 90%, at least 95%, at least 96%, at least 97%, at least 98%, at least 99% or 100%, sequence identity to the VL domain of antibody AE2P (SEQ ID NO: 10).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody AE2P in IgG₁ format (SEQ ID NO: 13).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody AE2P in IgG_{2A} format (SEQ ID NO: 17).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody AE2P in IgG₄format (SEQ ID NO: 22).

The antibody molecule may comprise a light chain which has at least 70%, more preferably at least 75%, at least 80%, at least 85%, at least 90%, at least 95%, at least 96%, at least 97%, at least 98%, at least 99% or 100%, sequence identity to the light chain of antibody AE2P (SEQ ID NO: 14).

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25 3389-3402) may be used.

Variants of the CDRs, VH domain, VL domain, heavy chain or light chain sequence disclosed herein comprising one or more, e.g. less than 20 alterations, less than 15 alterations, less than 10 alterations or less than 5 alterations, 4, 3, 2 or 1, amino acid alterations (addition, deletion, substitution and/or insertion of an amino acid residue) may also be employed in antibody molecules according to the invention. Suitable variants can be obtained by means of methods of sequence alteration, or mutation, and screening. Alterations may be made in one or more framework regions and/or one or more CDRs. In particular, alterations may be made in HCDR1, HCDR2 and/or HCDR3, or in one or more framework regions of the heavy or light chain of the antibody molecule.

As noted above, the antibody molecule may be a whole antibody or a fragment thereof, in particular an antigen-binding fragment thereof.

Preferably, the antibody molecule comprises or consists of a single-chain Fv (scFv), a small immunoprotein (SIP), a diabody, a single-chain diabody, a bispecific single-chain diabody (BiTE™) or a (whole) IgG molecule, such as an IgG1, IgG2A or IgG4 molecule.

Where the antibody molecule is an scFv, the VH and VL domains of the antibody are preferably linked by a 14 to 20 amino acid linker. For example, the VH and VL domains may be linked by an amino acid linker which is 14, 15, 16, 17, 18, 19, or 20 amino acid in length. Suitable linker sequences are known in the art and include the linker sequence set forth in SEQ ID NOs: 12 and 26.

In a preferred embodiment, the antibody molecule of the invention in scFv format comprises or consists of the sequence set forth in SEQ ID NO: 11 or SEQ ID NO: 25.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen-binding site: antigen-binding sites are formed by the association of the first domain of one polypeptide within the multimer with the second domain of another polypeptide within the multimer (WO94/13804; Holliger and Winter, 1997; Holliger et al., 1993).

In a diabody or single-chain diabody, a heavy chain variable domain (VH) is connected to a light chain variable domain (VL) on the same polypeptide chain. The VH and VL domains are connected by a peptide linker that is too short to allow pairing between the two domains. This forces pairing with the complementary VH and VL domains of another chain.

Where the antibody molecule is a diabody or single-chain diabody, the VH and VL domains are preferably linked by a 5 to 12 amino acid linker. For example, the VH and VL domains may be linked by an amino acid linker which is 5, 6, 7, 8, 9, 10, 11, or 12 amino acids in length. Preferably, the amino acid linker is 5 amino acids in length. Suitable linker sequences are known in the art and include the linker sequence set forth in SEQ ID NO: 21.

In a preferred embodiment, the antibody molecule of the invention in diabody format has the sequence set forth in SEQ ID NO: 19.

In a single-chain diabody, two sets of VH and VL domains are connected together in sequence on the same polypeptide chain. For example, the two sets of VH and VL domains may be assembled in a single-chain sequence as follows: (VH-VL)-(VH-VL), where the brackets indicate a set. The two sets of VH and VL domains are connected as a single-chain by a long or ‘flexible’ peptide linker. This type of peptide linker sequence is long enough to allow pairing of the VH and VL domains of the first set with the complementary VH and VL domains of the second set. Generally, a long or ‘flexible’ linker is 15 to 20 amino acids.

In a preferred embodiment, the antibody molecule of the invention in single-chain diabody (scDb) format has the sequence set forth in SEQ ID NO 65.

Where the antibody is a small immunoprotein (SIP) e.g. as described in (Li et al., (1997), Protein Engineering, 10:731-736), the VL domain of the scFv antibody is preferably linked to the CH4 domain of human IgE (Batista et al., (1996), J. Exp. Med., 184:2197-205) via a 2 to 20 amino acid linker, more preferably a 2 to 10 amino acid linker. Suitable linker sequences are known in the art and include the linker sequence set forth in SEQ ID NO: 16.

In a further preferred embodiment, the antibody molecule of the invention in SIP format has the sequence set forth in SEQ ID NO: 15.

### Bispecific Binding Molecules

An antibody molecule of the present invention may form part of a bispecific binding molecule, such as a bispecific antibody molecule. In some embodiments, the bispecific binding molecule comprises an antigen-binding site which binds a tumor associated antigen, e.g. fibroblast activation protein (FAP), the ED-A, ED-B, or IIICS splice isoform of fibronectin, CAIX, a splice isoform of tenascin-C such as the A1, A2, B, C or D isoform of tenascin-C, Mucin-16, PSMA or CEA. In a preferred embodiment, the bispecific binding molecule comprises an antigen-binding site which binds CEA. In an alternative preferred embodiment, the bispecific binding molecule comprises an antigen-binding site which binds FAP. In one embodiment, the second antigen-binding site of the bispecific binding molecule may not bind to PSMA. In this embodiment, the second antigen-binding site may bind FAP, the ED-A, ED-B, or IIICS splice isoform of fibronectin, CAIX, the A1, A2, B, C or D isoform of tenascin-C, Mucin-16, or CEA.

In addition, or alternatively, the bispecific binding molecule may comprise an antigen-binding site which binds a second T cell antigen, e.g. CD3.

A bispecific binding molecule according to the present invention is preferably a bispecific antibody, and may be selected from IgG-appended antibodies with an additional antigen-binding moiety (e.g. IgG-(scFv)₂ or IgG-(scFv)), and small recombinant bispecific antibody formats (e.g. bispecific T-cell engager (BiTE™) or scDb-scFv). Preferably, the bispecific antibody comprises a specificity against a tumor associated antigen, e.g. CEA or FAP.

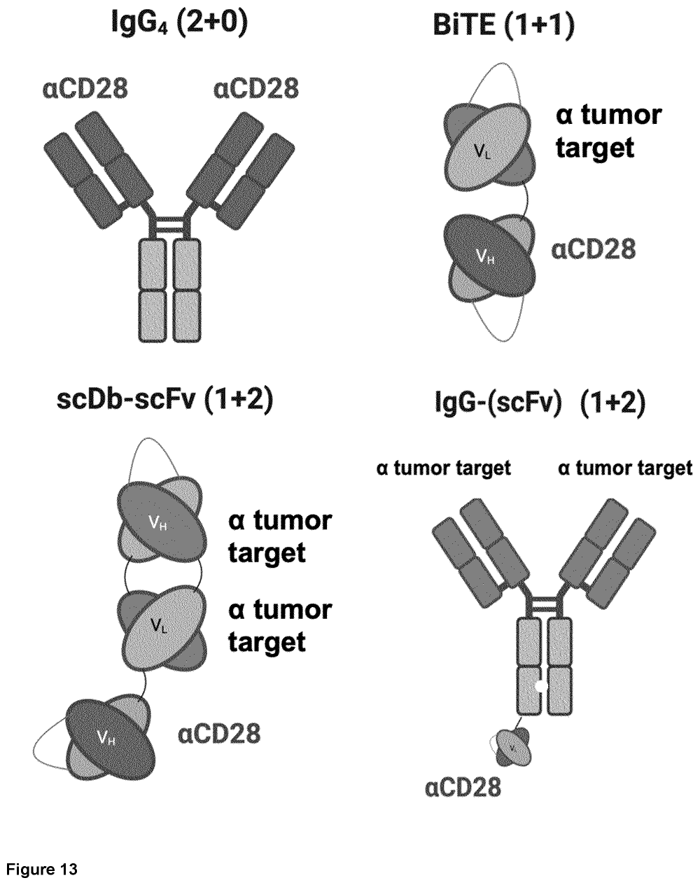

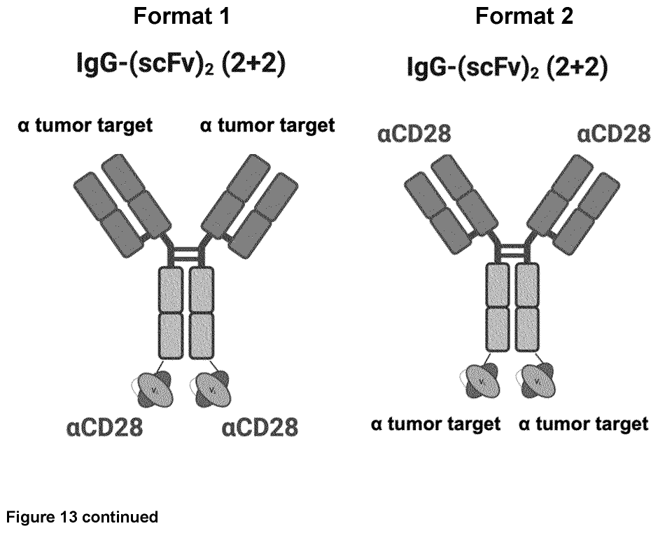

A BiTE™ is a single-chain diabody comprising two different sets of VH and VL domains, creating a bispecific single-chain diabody. The sequence of the AE2P antibody and the anti-FAP antibody 7NP2 in bispecific T cell engager (BiTE™) format is shown in SEQ ID NO: 23. An example of an antibody in BiTE™ format is shown in FIG. 13 (see BiTE 1+1).

An scDb-scFv is a single-chain diabody which binds a first target conjugated to an scFv which binds as second target. The scFv may be conjugated, e.g. via an amino acid linker, to a VL domain of the single-chain diabody. In an scDb-scFv, the AE2P antibody may be in scDb or scFv format. Thus, in one example, the AE2P antibody is in scFv format and the scDb binds a tumour associated antigen. Alternatively, the AE2P antibody may be in scDb format and the scFv may bind a tumor associated antigen. An example of an antibody in scDb-scFv format is shown in FIG. 13 (see scDb-scFv (1+2)).

An IgG-appended antibody comprising an additional antigen-binding moiety according to the present invention preferably comprise an IgG molecule and one or two scFvs. The scFvs are preferably conjugated to the C-terminus of the heavy chain(s) of the IgG molecule via an amino acid linker. Where the IgG-appended antibody comprises a single scFv molecule conjugated to the C-terminus of one of the two heavy chains of the IgG molecule, the C-terminus of the other heavy chain is preferably free, i.e. unconjugated. This antibody format is also referred to as IgG-(scFv). An IgG-appended antibody which comprises an scFv molecule conjugated to the C-terminus of both heavy chains of an IgG molecule, is also referred to as IgG-(scFv)₂. These antibody formats are shown in FIG. 13 (see IgG-(scFv) 1+2 and IgG-(scFv)₂ 2+2 formats). In an IgG-appended antibody according to the present invention, the AE2P antibody may be in IgG or scFv format. Thus, in one example, the AE2P antibody is in IgG format and one or both of the heavy chains of the AE2P antibody in IgG format are conjugated to an scFv that binds a tumour-associated antigen. Alternatively, the IgG-appended antibody comprises an IgG that binds a tumour-associated antigen and one or both of the heavy chains of said IgG are conjugated to the AE2P antibody in scFv format. The sequences of the heavy chain and the light chain of an anti-human CEA antibody and the AE2P antibody in IgG-(scFv)₂ format, wherein the anti-human CEA antibody is in IgG format and the AE2P antibody is in scFv format (format 1, FIG. 13), are shown in SEQ ID NO: 61 and 62 respectively. The sequences of the heavy chain and the light chain of the AE2P antibody and an anti-human CEA antibody in IgG-(scFv)₂ format, wherein the AE2P antibody is in IgG format and the anti-human CEA antibody is in scFv format (format 2, FIG. 13), are shown in SEQ ID NO: 63 and 64 respectively.

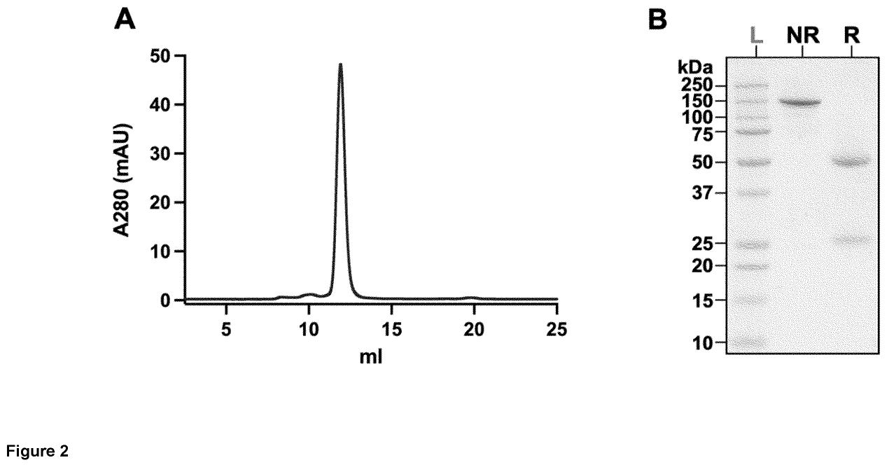

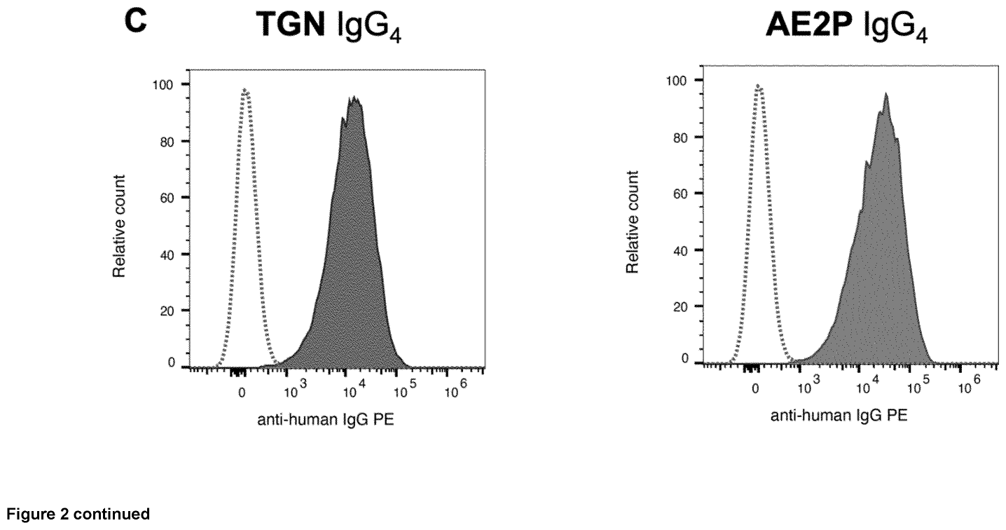

Further examples of bispecific binding molecules can be found in Kontermann 2012 (page 186 FIG. 2) the content of which is incorporated herein by reference.

### Conjugate

Conjugates of the invention comprise an antibody molecule of the invention and a therapeutic or diagnostic agent. The therapeutic agent may be a pro-inflammatory agent, a radioisotope, a photosensitizer, an enzyme, or a hormone.

Pro-inflammatory cytokines which may be conjugated to an antibody molecule of the invention include interleukin-2 (IL2), interleukin-12 (IL12), interleukin-15 (IL15), interferon (IFN), such as IFNγ, and tumor necrosis factor (TNF), such as TNFα, as well as mutants or variants thereof.

A therapeutic agent may be conjugated to the N-terminus or C-terminus of the antibody molecule or both. Where a therapeutic agent is conjugated to both the N-terminus and the C-terminus of the antibody molecule, the therapeutic agents may be the same or different but preferably are different. Where the therapeutic agent is conjugated to the N-terminus of the antibody molecule, the C-terminus may be “free”, i.e. not conjugated to another moiety. Similarly, where the therapeutic agent is conjugated to the C-terminus of the antibody molecule, the N-terminus may be “free”, i.e. not conjugated to another moiety.

A diagnostic agent conjugated to the antibody molecule of the invention may be a detectable label, such as a radioisotope, e.g. a non-therapeutic radioisotope.

Radioisotopes which may be conjugated to an antibody molecule of the invention include isotopes such as ^{94m}Tc, ^{99m}Tc, ¹⁸⁶Re, ¹⁸⁸Re, ²⁰³Pb, ⁶⁷Ga, ⁶⁸Ga, ⁴⁷Sc, ¹¹¹ In, ⁹⁷Ru, ⁶²Cu, ⁶⁴Cu, ⁸⁶Y, ⁸⁸Y, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At and ²²⁵Ac. Preferably, positron emitters, such as ₁₈F and ₁₂₄I, or gamma emitters, such as ^{99m}Tc, ¹¹¹ In and ¹²³I, are used for diagnostic applications (e.g. for PET), while beta-emitters, such as ¹³¹I, ⁹⁰Y and ¹⁷⁷Lu, are preferably used for therapeutic applications. Alpha-emitters, such as ²¹¹At and ²²⁵Ac may also be used for therapy. In one example, the antibody molecule may be conjugated to ¹⁷⁷Lu, ¹³¹I, or ⁹⁰Y.

The antibody molecule may be conjugated with the therapeutic agent by means of a peptide bond or linker as described herein. Other means for conjugation include chemical conjugation, especially cross-linking using a bifunctional reagent (e.g. employing DOUBLE-REAGENTS™ Cross-linking Reagents Selection Guide, Pierce).

### Linkers

The antibody molecule, e.g. scFv or IgG, and the therapeutic or diagnostic agent or molecule, may be connected to each other directly, for example through any suitable chemical bond, but preferably are connected via a peptide linker. The chemical bond may be, for example, a covalent or ionic bond. Examples of covalent bonds include peptide bonds (amide bonds) and disulphide bonds.

Where the therapeutic or diagnostic agent is connected to the antibody molecule via a peptide linker, the peptide linker may be a short (2-30, preferably 10-20) residue stretch of amino acids.

Suitable examples of peptide linker sequences are known in the art. One or more different linkers may be used.

Where the antibody molecule and therapeutic or diagnostic agent are connected via a peptide bond or peptide linker, the conjugate may be produced (secreted) as a single chain polypeptide, such as a fusion protein.

### Methods of Treatment

An antibody molecule or conjugate of the invention may therefore be for use as a medicament.

In particular, the antibody molecule or conjugate may be for use in a method of treatment (which may include prophylactic treatment) of the human or animal body.

Also provided is a method of treating a disease or disorder in a patient, wherein the method comprises administering to the patient a therapeutically effective amount of the antibody molecule or conjugate.

Further provided is the use of the antibody molecule or conjugate in the manufacture of a medicament for use in the treatment of a disease or disorder in a patient.

Treatment may be any treatment or therapy in which some desired therapeutic effect is achieved, for example, the inhibition or delay of the progress of the disease or disorder, and includes a reduction in the rate of progress, a halt in the rate of progress, amelioration of the disease or disorder, cure or remission (whether partial or total) of the disease or disorder, preventing, ameliorating, delaying, abating or arresting one or more symptoms and/or signs of the disease or disorder or prolonging survival of an individual or patient beyond that expected in the absence of treatment.

Treatment as a prophylactic measure (i.e. prophylaxis) is also included. For example, an individual susceptible to or at risk of the occurrence or re-occurrence of a disease or disorder may be treated as described herein. Such treatment may prevent or delay the occurrence or re-occurrence of the disease or disorder in the individual.

A method of treatment as described may comprise administering at least one further treatment to the individual in addition to the antibody molecule or conjugate. The antibody molecule or conjugate may thus be administered to an individual alone or in combination with one or more other treatments for the disease or disorder in question. Where the antibody molecule or conjugate is administered to the individual in combination with another treatment, the additional treatment may be administered to the individual concurrently with, sequentially to, or separately from the administration of the antibody molecule or conjugate. Where the additional treatment is administered concurrently with the antibody molecule or conjugate, the antibody molecule or conjugate and additional treatment may be administered to the patient as a combined preparation. For example, the additional therapy may be a known therapy or therapeutic agent for the disease or disorder to be treated. For example, an antibody molecule or conjugate of the invention may be employed in a method of treatment as described herein in combination with a CD3 agonist, for example an antibody which binds human CD3.

Exemplary further therapeutic agents that may be combined with or administered in association with an antibody molecule of the present invention include, e.g., chemotherapy (e.g., anti-cancer chemotherapy, for example, paclitaxel, docetaxel, vincristine, cisplatin, carboplatin, or oxaliplatin), radiation therapy, a checkpoint inhibitor that targets PD-1 (e.g., an anti-PD-1 antibody such as pembrolizumab, nivolumab, or cemiplimab (see U.S. Pat. No. 9,987,500)), CTLA-4, LAG3, or TIM3, a costimulatory agonist antibody that targets e.g. GITR, OX40, or 4-1 BB, and other costimulatory CD28 bispecific antibodies.

The disease to be treated using an antibody molecule or conjugate of the invention may be cancer, as well as other tumors and neoplastic conditions.

Exemplary cancers include any type of solid or non-solid cancer or malignant lymphoma and especially liver cancer, lymphoma, leukaemia (e.g. acute myeloid leukaemia), sarcomas, skin cancer, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic.

The disease to be treated using an antibody molecule or conjugate of the invention may be a chronic infectious disease. Exemplary chronic infectious diseases include chronic hepatitis B infection (HBV), human immunodeficiency virus (HIV) infection, and tuberculosis.

Autoimmune diseases which may be treated using an antibody molecule or conjugate of the invention herein include lupus erythematosus, rheumatoid arthritis, and psoriatic arthritis.

An inflammatory or autoimmune disease which may treated using an antibody molecule or conjugate of the invention is inflammatory bowel disease (IBD), such Crohn's disease or ulcerative colitis.

The disease to be treated using an antibody molecule or conjugate of the invention may be a transplantation-associated disease. Transplantation-associated diseases which may be treated using an antibody molecule or conjugate of the invention herein include transplant rejection, such as acute transplant rejection and chronic transplant rejection. Chronic transplant rejection includes graft-versus-host disease (GvHD).

### Methods of Detection or Diagnosis

The antibody molecules and conjugates are expected to be suitable for detecting CD28 in vivo and in vitro, and thus find application in the imaging, detection and diagnosis of disease characterised by, or associated with, expression of CD28.

The present invention therefore also relates to the use of an antibody molecule or conjugate of the invention for detecting CD28 on their cell surface, either in vitro or in vivo. The conjugate preferably comprises a detectable label to aid detection. Alternatively, binding of the antibody molecule to CD28 may be detected using a secondary antibody or other detection reagent. Where the antibody molecule is conjugated to a radioisotope, binding of the antibody molecule to CD28 in the patient may be detected using scintigraphy.

Also provided is an in vitro method for detecting CD28, the method comprising incubating the antibody molecule or conjugate with a sample obtained from an individual, e.g. a human patient, and detecting binding of the antibody molecule or conjugate to the sample, e.g. T-cells present in the sample, wherein binding of the antibody molecule or conjugate to the sample indicates the presence of CD28. Methods for determining binding of an antibody molecule or antigen to a sample are known in the art and include, for example, ELISAs, flow cytometry, and immunostaining of tissue samples.

Further provided is the antibody molecule or conjugate for use in a method of detecting CD28 in vivo, the method comprising administering the antibody molecule or conjugate to an individual, e.g. a human patient.

### Pharmaceutical Compositions

Whilst an antibody molecule or conjugate may be administered alone, antibody molecules and conjugates will typically be administered in the form of a pharmaceutical composition. Thus, a further aspect of the present invention relates to a pharmaceutical composition comprising at least one antibody molecule or conjugate of the invention and at least one other component, such as a pharmaceutically acceptable excipient. A method comprising formulating an antibody molecule or conjugate into a pharmaceutical composition is also provided.

Pharmaceutical compositions may comprise, in addition to the antibody molecule or conjugate, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g. by injection, the pharmaceutical composition comprising the antibody molecule or conjugate may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required, including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagine, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

In some embodiments, the antibody molecules or conjugates may be provided in a lyophilised form for reconstitution prior to administration. For example, lyophilised antibody molecules or conjugates may be re-constituted in sterile water and mixed with saline prior to administration to an individual.

Administration may be in a “therapeutically effective amount”, this being sufficient to show benefit to an individual. The actual amount administered, and rate and time-course of administration, will depend on the nature and severity of the disease or disorder being treated, the particular individual being treated, the clinical condition of the individual, the cause of the disorder, the site of delivery of the composition, the type of antibody molecule or conjugate, the method of administration, the scheduling of administration and other factors known to medical practitioners. Prescription of treatment, e.g. decisions on dosage etc., is within the responsibility of general practitioners and other medical doctors. Appropriate doses of antibody molecules are well known in the art (Ledermann et al., 1991; Bagshawe et al., 1991). Specific dosages indicated herein, or in the Physician's Desk Reference (2003) as appropriate for an antibody molecule being administered, may be used. Appropriate doses for conjugates are also known or can be determined. For example, a therapeutically effective amount or suitable dose of an antibody molecule or conjugate can be determined by comparing in vitro activity and in vivo activity in an animal model, such as a domestic dog, a pig, or a sheep. Methods for extrapolation of effective dosages in domestic dogs, pigs and sheep, as well as other test animals to humans are known. The precise dose will depend upon a number of factors, including whether the size and location of the area to be treated, and the precise nature of the antibody molecule or conjugate.

Treatments may be repeated at daily, twice-weekly, weekly or monthly intervals, at the discretion of the physician. The treatment schedule for an individual may be dependent on the pharmacokinetic and pharmacodynamic properties of the antibody molecule or conjugate, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Suitable formulations and routes of administration are described above.

A pharmaceutical composition may be administered alone or in combination with other treatments, either simultaneously or sequentially dependent upon the condition to be treated.

### Kits

Another aspect of the invention provides a therapeutic kit for use in the treatment of a disease or disorder comprising an antibody molecule or conjugate as described herein. The components of a kit are preferably sterile and in sealed vials or other containers.

A kit may further comprise instructions for use of the components in a method described herein. The components of the kit may be comprised or packaged in a container, for example a bag, box, jar, tin or blister pack.

### Nucleic Acids, Vectors, Host Cells and Methods of Production

Provided is an isolated nucleic acid molecule encoding an antibody molecule or conjugate of the invention. Nucleic acid molecules may comprise DNA and/or RNA and may be partially or wholly synthetic.

An isolated nucleic acid molecule may be used to express an antibody molecule or conjugate of the invention. The nucleic acid will generally be provided in the form of an expression vector. Another aspect of the invention thus provides an expression vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate in the context.

A nucleic acid molecule or expression vector as described herein may be introduced into a host cell. Techniques for the introduction of nucleic acid or vectors into host cells are well established in the art and any suitable technique may be employed. A range of host cells suitable for the production of recombinant antibody molecules and conjugates are known in the art, and include bacterial, yeast, insect or mammalian host cells. A preferred host cell is a mammalian cell, such as a CHO, NS0, or HEK cell, for example a HEK293 cell.

Another aspect of the invention provides a method of producing an antibody molecule, or conjugate, comprising expressing a nucleic acid encoding the antibody molecule, or conjugate, in a host cell and optionally isolating and/or purifying the antibody molecule, or conjugate, thus produced. Methods for culturing host cells are well-known in the art. The method may further comprise isolating and/or purifying the antibody molecule or conjugate. Techniques for the purification of recombinant antibody molecules, or conjugates, are well-known in the art and include, for example HPLC, FPLC, or affinity chromatography, e.g. using Protein A or Protein L. In some embodiments, purification may be performed using an affinity tag on antibody molecule. The method may also comprise formulating the antibody molecule, or conjugate, into a pharmaceutical composition, optionally with a pharmaceutically acceptable excipient or other substance as described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### EXAMPLE 1—Cloning of Human CD28 and Isolation of AE2P by Phage Display

### 1.1 Expression Procedure

A human CD28-ECD (extracellular domain) recombinant fragment containing a C-terminal Avi tag, His6 tag and Fc tag (“CD28-ECD” also interchangeably referred to as “CD28-Fc” and “CD28-Fc fusion protein” herein) was expressed using transient gene expression (TGE) in CHO-S cells. For 1 mL of production 4×10⁶ CHO-S cells in suspension were centrifuged and resuspended in 1 mL of a suitable medium. 0.9 μg of plasmid DNA followed by 2.5 μg polyethylene imine (PEI; 1 mg/mL solution in water at pH 7.0) per million cells were then added to the cells and gently mixed. The transfected culture was incubated in a shaker incubator at 31° C. for 6 days. The protein fragment was purified from the cell culture medium by using protein A affinity chromatography and then dialyzed into PBS buffer and stored at −80° C. The amino acid sequence of the CD28-Fc fusion protein is shown in SEQ ID NO: 66.

### 1.2 Antigen Characterization

The human CD28-ECD fusion protein was analyzed by SDS-PAGE and by size exclusion chromatography using a Superdex 200 increase (S200i) 10/300 GL column on an ÄKTA FPLC. Binding of the recombinant antigen was validated by ELISA against anti-CD28 antibodies.

### 1.3 Antigen Biotinylation

The purified human CD28-ECD fusion protein was site specifically biotinylated using BirA (E.coli biotin ligase). The protein was first dialyzed in BirA buffer (100 mM Tris PH 7.5, 200 mM Nacl, 5 mM MgCl). The biotinylation reaction was performed for 24 hours by adding 1 mg of protein, 28 μl of 40 mM Biotin, 43 μg of BirA, 70 uL of 0.5M ATP, and protease inhibitors. The next day, the protein was purified by size exclusion chromatography and dialyzed back into PBS buffer.

### 1.4 Phage Display Selection

The biotinylated human CD28-ECD fusion protein was used to perform biopanning with streptavidin magnetic beads. Briefly, the biotinylated human CD28-ECD fusion protein (final concentration 120 pmol) was incubated with 800 μL of a pre-blocked phage display library for 30 minutes. After several washes with PBS buffer, bound phages were eluted by changing the pH using triethylamine. Isolated phages were then amplified in E. coli strain TG-1 and precipitated from the supernatant with polyethylene glycol.

After two rounds of biopanning, clones were screened by ELISA. Avidin-coated ELISA plates were incubated with biotinylated human CD28-ECD fusion protein. To exclude phages that bound to Fc, biotinylated human IgG was also used in parallel to coatAvidin-coated ELISA plates. The supernatants of selected induced monoclonal clones of the E. coli TG-1 cultures expressing scFv antibody fragments were added to both ELISA plates and bound scFvs were detected using the anti-c-myc antibody 9E10 followed by the use of an anti-mouse IgG-horseradish peroxidase (HRP) conjugate.

### EXAMPLE 2—Biochemical Characterization

### 2.1 In Vitro Characterization of Antibody AE2P in scFv Format

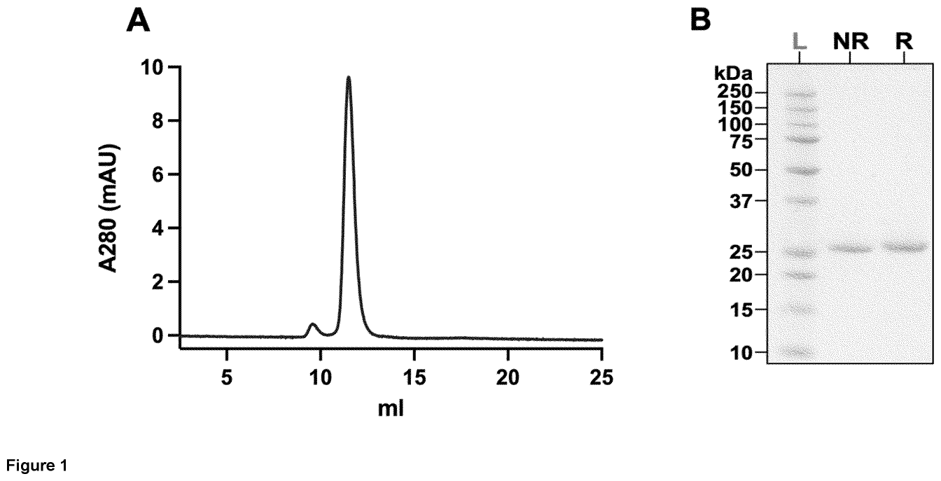

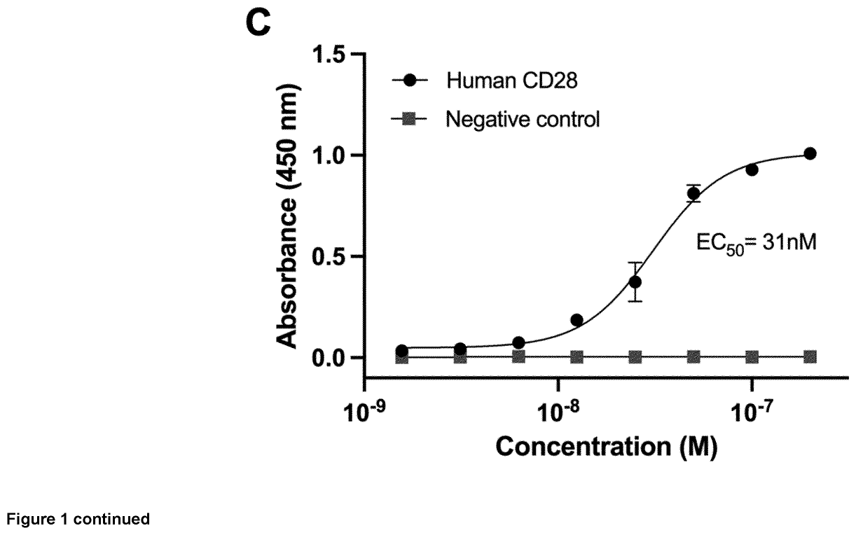

AE2P scFv was produced in E. coli strain TG-1. A TG-1 culture was grown at 37° C. in 2×TY/100 μg/ml ampicillin. At OD600=0.5, 1 mM isopropyl-thio-galactopyranoside (IPTG) was added to induce expression of the scFv; the culture was incubated on a bacterial incubator shaking at 175 rpm at 30° C. overnight. The culture was then centrifuged, and the supernatant purified from the cell culture medium by protein A affinity chromatography and then dialyzed against PBS and stored in PBS at −80° C. The AE2P scFv was then characterized and purified by size exclusion chromatography using an S75i 10/300 GL column on an ÄKTAFPLC (FIG. 1A). SDS-PAGE analysis was also performed with 4-12% Bis-Tris gel under reducing and non-reducing conditions (FIG. 1B). Titration ELISA was performed by coating either biotinylated CD28-ECD fusion protein or biotinylated non-specific protein on streptavidin wells. After blocking with 2% milk-PBS, various concentrations of AE2P scFv were added and the signal was detected using mouse anti-myc tag followed by anti-mouse HRP. A sigmoidal curve was plotted at the different concentrations (FIG. 1C). These results showed that the AE2P scFv had the expected molecular weight under reducing and non-reducing conditions and was eluted from the SEC column at 11.5 ml, with the single peak observed by SEC indicating excellent purity. The titration ELISA shows an approximate EC₅₀ of 31 nM confirming the binding of the AE2P scFv to CD28.

### 2.2 In Vitro Characterization of the AE2P Antibody in IgG₄ format

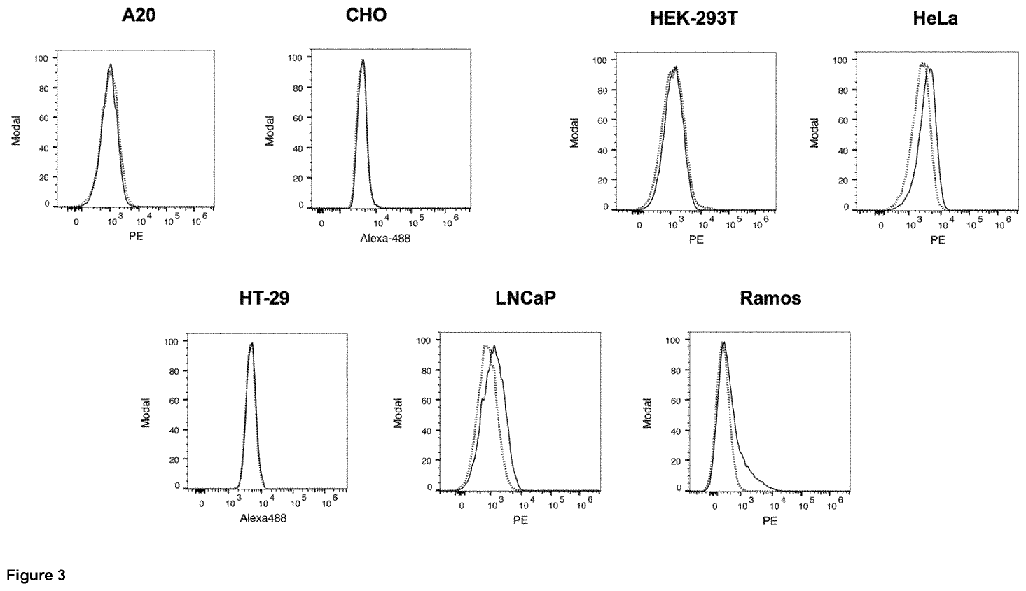

The AE2P antibody in IgG₄ format was produced by the same method as described above. Specifically, the AE2P IgG₄ was cloned, expressed using transient gene expression (TGE) in CHO-S cells, purified by protein A affinity chromatography, dialyzed, and stored in PBS. AE2P IgG₄ was then characterized by size exclusion chromatography using an S200i 10/300 GL column on an ÄKTA FPLC (FIG. 2A). SDS-PAGE analysis was also performed with 4-12% Bis-Tris gel under reducing and non-reducing conditions (FIG. 2B). Flow cytometry analysis on primary human T cells was performed by staining with 50 nM of AE2P IgG₄, TGN1412 IgG₄ (as defined in SEQ ID Nos: 27 and 28; U.S. Pat. No. 8,709,414), or isotype IgG₄. Cells were first blocked for 30 minutes in FACS buffer (PBS, 2% FBS and 2 mM EDTA). After incubating for 1 hour with primary antibodies, cells were washed twice with FACS buffer and secondary anti-human PE antibodies were added to detect the fluorescence shift in case of binding (FIG. 2C). Zombie near infrared (NIR) was used to discriminate live from dead cells. Flow cytometry analysis on various cell lines was performed by staining with 50 nM of AE2P IgG₄ or isotype IgG₄ similar to that mentioned above. Secondary anti-human PE or anti-human Alexa 488 antibodies were added to detect the fluorescence shift in case of binding (FIG. 3). These results showed that the AE2P IgG₄ had the expected molecular weight under reducing and non-reducing conditions and was eluted from the SEC column at 11.9 mL, with the single peak observed by SEC indicating excellent purity. The flow cytometric analysis in FIG. 2C shows binding of both AE2P IgG₄ and TGN1412 IgG4 to primary human T cells expressing CD28. The flow cytometric analysis in FIG. 3 shows that AE2P IgG₄ binds specifically to primary human T cells and not to other cell lines which do not express CD28.

### EXAMPLE 3—Co-Stimulation Assay in Combination With the Anti-CD3 Monoclonal Antibody “OKT3”

### 3.1 Cell Proliferation in Combination With OKT3

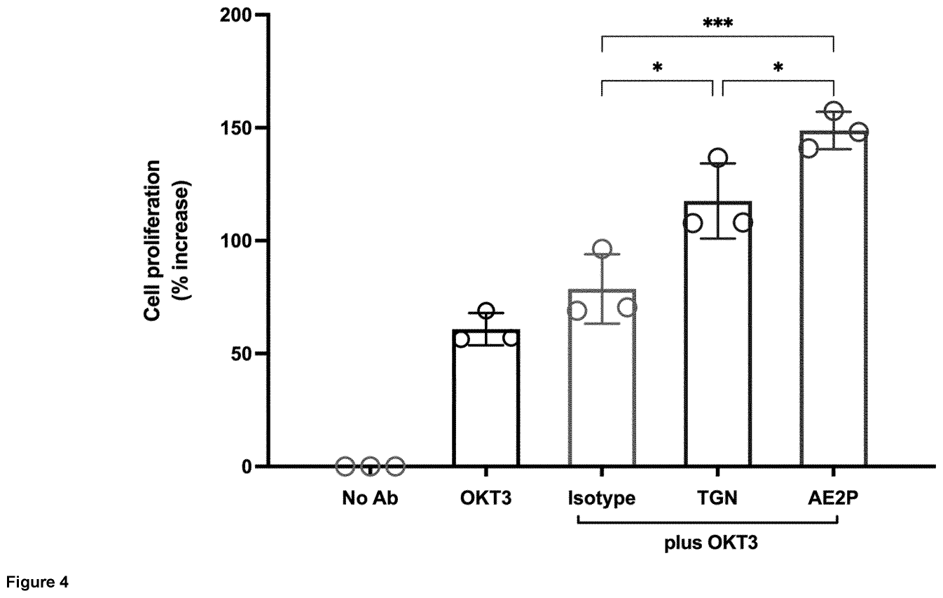

The co-stimulatory effect of AE2P and TGN1412 antibodies was tested by comparing the degree of cell proliferation induced by each antibody in the presence of CD3 stimulation. Specifically, OKT3 (Muromonab-CD3™) is an activating monoclonal antibody against CD3 receptor. OKT3 IgG₄ was coated on a 6 well plate (1 μg per 1 mL in 2 mL) for 2 hours at 37° C. in a cell culture incubator. Purified human PBMCs from a healthy donor (0.5 million per 1 mL in 3 mL) were added to the 6 well plate (in triplicate per experimental condition). AE2P IgG₄, TGN1412 IgG₄ or isotype IgG₄ at a concentration of 5 μg per 1 mL was added in solution to each experimental condition. Human PBMCs without added antibodies were also included as a control. The plate was incubated for 3 days at 37° C. in a cell culture incubator. For cell proliferation, 200 μl from each triplicate was added to a 96 well plate and 20 μl of CellTiter 96 Aqueous One Solution Cell Proliferation assay was added and incubated for 2 hours at 37° C. in a cell culture incubator. Absorbance at 490 nm was measured and percentage increase in cell proliferation (normalized to control condition) was plotted (FIG. 4). These results show the co-stimulatory effect on proliferation of human PBMCs generated by the anti-CD28 antibodies (AE2P IgG₄ and TGN1412 IgG4) in combination with an anti-CD3 antibody (OKT3). In particular, the increase in cell proliferation in the presence of AE2P IgG₄ is shown to be greater than that achieved with TGN1412 IgG₄, indicating that a greater co-stimulatory effect is generated by AE2P IgG₄.

### 3.2 Cytokine Release in Combination With OKT3

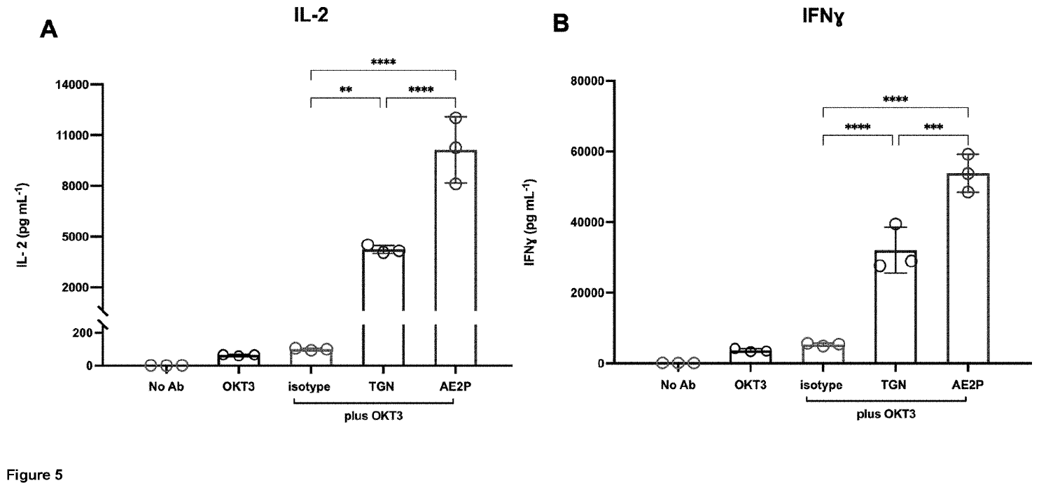

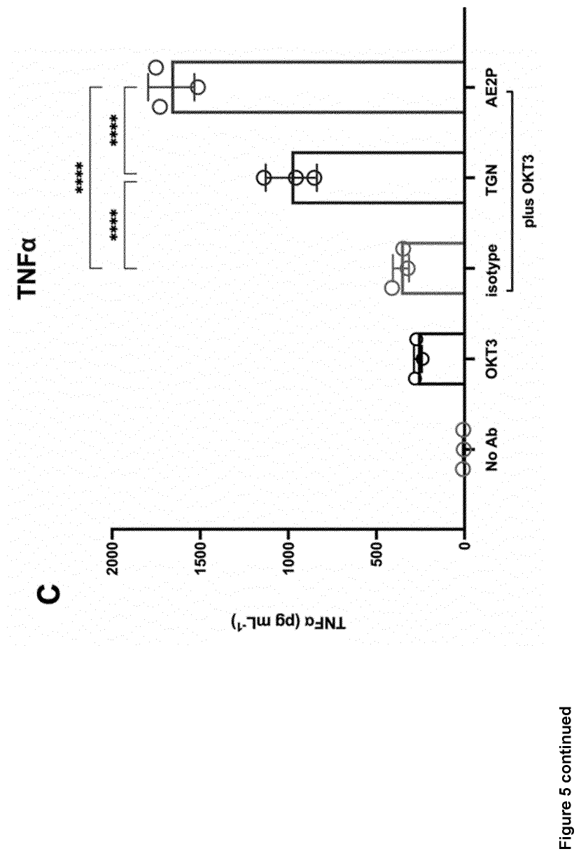

The co-stimulatory effect of AE2P and TGN1412 antibodies was further tested by comparing the degree of cytokine release induced by each antibody in the presence of CD3 stimulation. Specifically, Cells from the assay mentioned above (section 3.1) were spun down at 500 g for 10 minutes and the supernatant was transferred and stored at −20° C. Cytokine release was measured by ELISA for IL-2 (FIG. 5A), IFN gamma (FIG. 5B) and TNF alpha (FIG. 5C). These results show the increase in IL-2, IFN-gamma, and TNF-alpha secretion from human PBMCs when using anti-CD28 antibodies (AE2P IgG4 and TGN1412 IgG₄) in combination with an anti-CD3 antibody (OKT3). For all three cytokines, the increase in secretion in the presence of AE2P IgG₄ was greater than that in the presence of TGN1412 IgG₄, indicating that AE2P IgG₄ provides a greater co-stimulatory effect.

EXAMPLE 4—Super-Agonism Assay of AE2P

### 4.1 Cell Proliferation

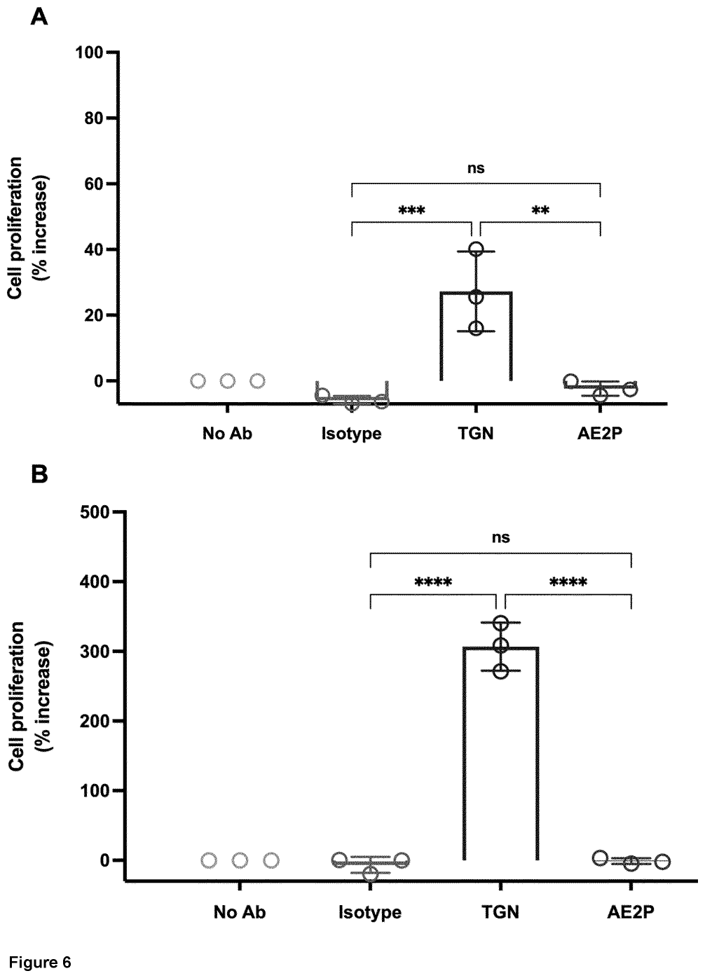

The AE2P and TGN1412 antibodies were tested for super-agonistic activity by comparing the degree of cell proliferation induced by each antibody in the absence of CD3 stimulation. Specifically, AE2P antibody in IgG₄ format, TGN1412 antibody in IgG₄ format), or isotype IgG₄ were coated on a 6 well plate (3 μg per 1 mL in 2 mL) for 2 hours at 37° C. in cell culture incubator. Purified human PBMCs from a healthy donor (0.5 million per 1 mL in 3 mL) were added to the 6 well plate (in triplicate per experimental condition). Human PBMCs without added antibodies were also included as a control. The plate was incubated for 3 days at 37° C. in cell culture incubator. The cell proliferation assay was performed as mentioned above (section 3.1) after 3 days (FIG. 6A) and 5 days (FIG. 6B). These results show that, in contrast to TGN1412 IgG₄, AE2P IgG₄ does not induce proliferation of human PBMCS in the absence of CD3 stimulation, demonstrating that AE2P does not have super-agonist activity.

### 4.2 Cytokine Release

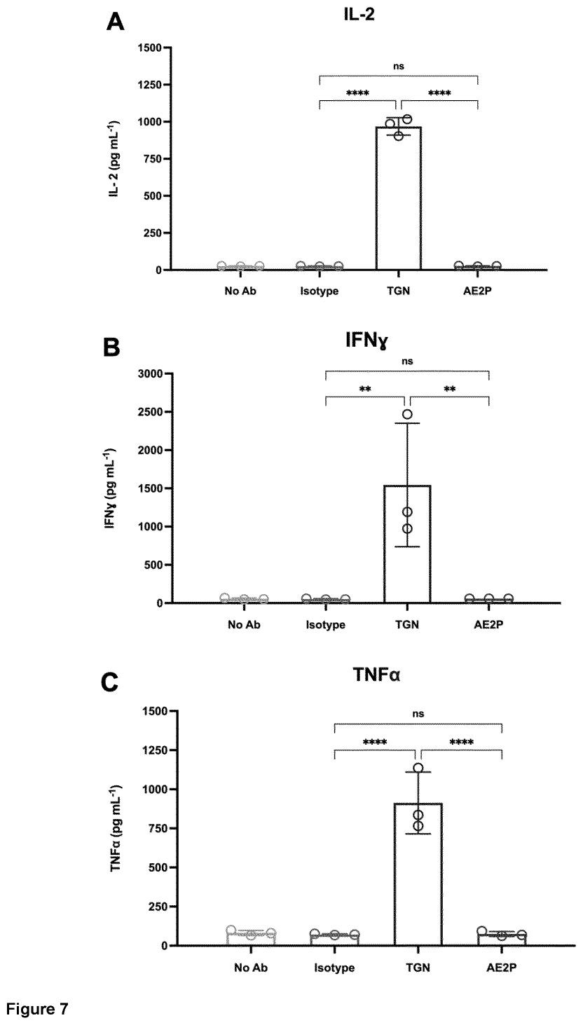

The AE2P and TGN1412 antibodies were further tested for super-agonist activity by comparing the degree of cytokine release induced by each antibody in the absence of CD3 stimulation. Specifically, cells from the assay mentioned above (section 4.1) were spun down at 500 g for 10 minutes and the supernatant was transferred and stored at −20° C. Cytokine release was measured after 3 days by ELISA for IL-2 (FIG. 7A), IFN gamma (FIG. 7B) and TNF alpha (FIG. 7C). These results show that AE2P IgG₄, in contrast to TGN1412 IgG₄, did not induce secretion of any of the three cytokines (IL-2, IFN gamma, and TNF alpha by human PBMCS, confirming that this antibody does not have super-agonist activity.

### 4.3 Expression of Activation Markers

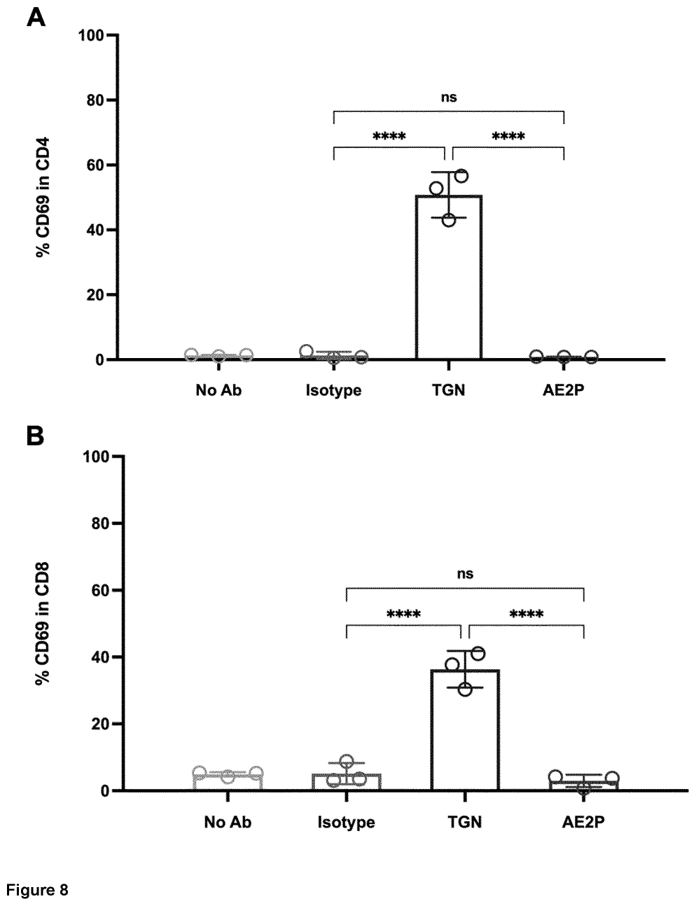

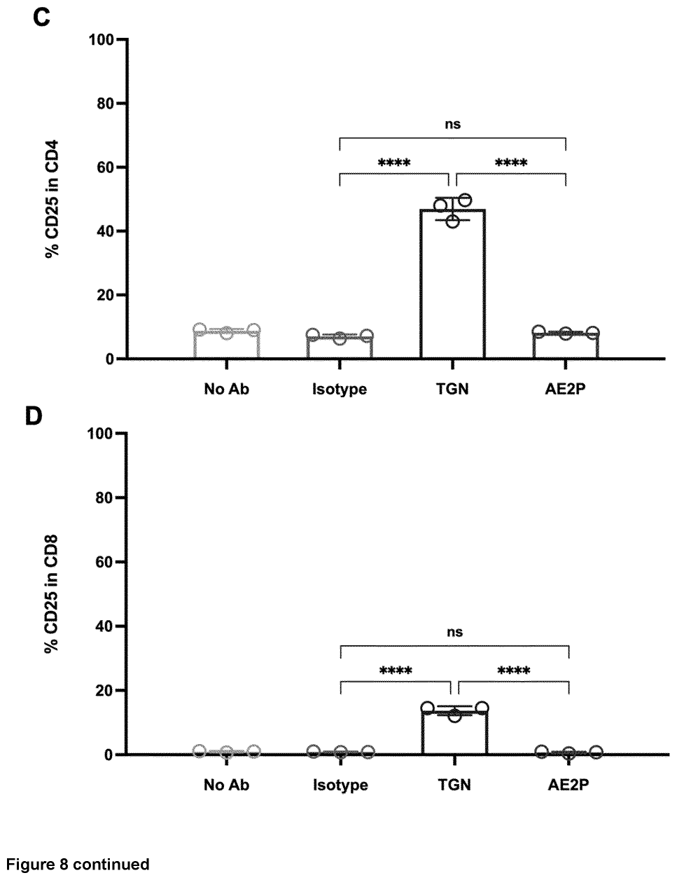

The AE2P and TGN1412 antibodies were further tested for super-agonist activity by comparing the degree of activation marker upregulation induced by each antibody in the absence of CD3 stimulation. Specifically, cells from the assay mentioned above (section 4.1) were spun down at 500 g for 10 minutes. Expression of activation markers (CD69 and CD25) after 3 days was assessed by flow cytometry using the following antibody panel; CD4-FITC, CD8-BV421, CD69-PE and CD25-Alexa 647. Zombie NIR was added to discriminate live cells from dead cells. Data was plotted as percentage of CD69 expression in CD4 positive cells (FIG. 8A), percentage of CD69 expression in CD8 positive cells (FIG. 8B), percentage of CD25 expression in CD4 positive cells (FIG. 8C) and percentage of CD25 expression in CD8 positive cells (FIG. 8D). These results show that AE2P IgG₄, in contrast to TGN1412 IgG₄, did not induce upregulation of the activation markers CD69 and CD25 in CD4 positive T cells and CD8 positive T cells, confirming that this antibody does not have super-agonist activity.

### EXAMPLE 5—Cross-Reactivity of AE2P With Murine CD28 ECD

### 5.1 Binding of AE2P (IgG₄) to Human and Murine CD28 ECD

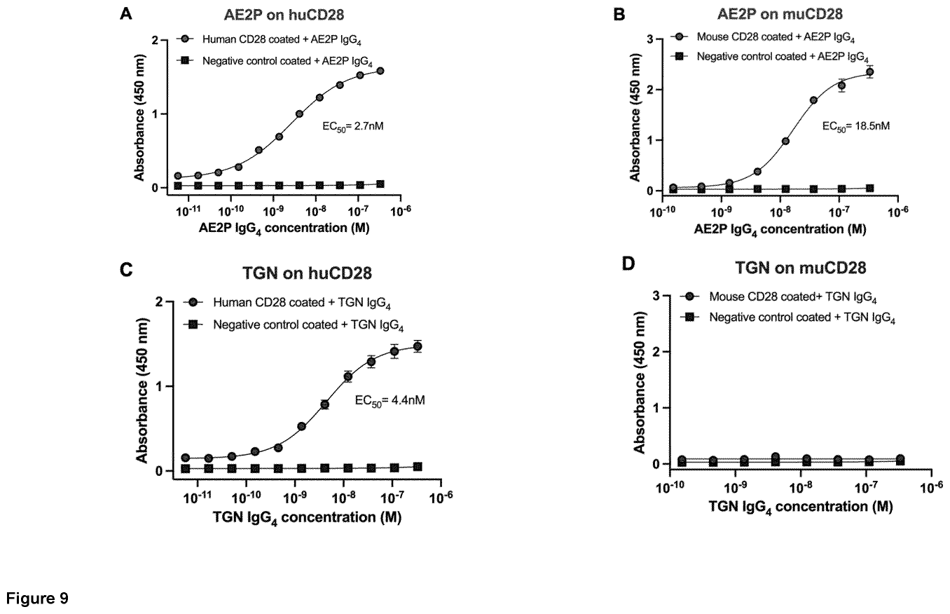

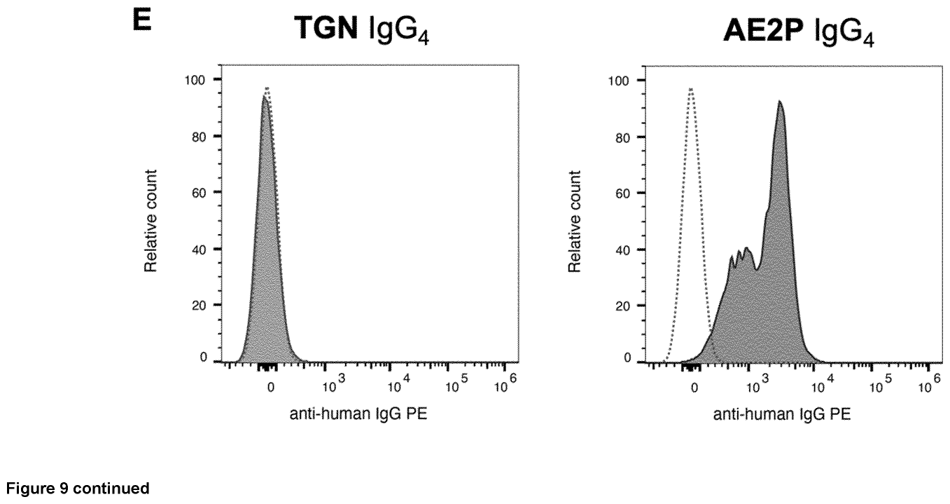

To test whether the AE2P antibody could also bind to murine CD28, titration ELISA was performed by coating either CD28-ECD protein or non-specific protein, both containing His6 Tag, on NiNTA coated plates. After blocking with 2% milk-PBS, various concentrations of FITC-conjugate AE2P IgG₄ were added, and the signal was detected using rabbit anti-FITC followed by goat anti-rabbit HRP. A sigmoidal curve was plotted at the different concentrations for binding to human CD28 (FIG. 9A) and for murine CD28 (FIG. 9B). These results demonstrate that AE2P IgG₄ is capable of binding to both human and murine CD28 Fc.

### 5.2 Binding of TGN1412 (IgG₄) to Human and Murine CD28 ECD

To test whether the TGN1412 antibody could also bind to murine CD28, titration ELISA was performed by coating either CD28-ECD protein or non-specific protein, both containing His6 Tag, on NiNTA coated plates. After blocking with 2% milk-PBS, various concentrations of FITC-conjugate TGN1412 IgG₄ were added, and the signal was detected using rabbit anti-FITC followed by goat anti-rabbit HRP. A sigmoidal curve was plotted at the different concentrations for binding to human CD28 (FIG. 9C) and for murine CD28 (FIG. 9D). These results demonstrate that TGN1412 IgG₄ binds to human CD28 Fc but is not capable of binding to murine CD28 Fc.

### 5.3 Binding to Primary Mouse T Cells

To further test whether the AE2P and TGN1412 antibodies could also bind to murine CD28 expressed on cells, flow cytometry analysis on primary mouse T cells was performed by staining with 50 nM of AE2P IgG₄, TGN1412 IgG₄ or isotype IgG₄. Cells were first blocked for 30 minutes in FACS buffer (PBS, 2% FBS and 2 mM EDTA). After incubating for 1 hour with primary antibodies, cells were washed twice with FACS buffer and secondary anti human PE antibody was added to detect the fluorescence shift in case of binding (FIG. 9E). Zombie NIR was used to discriminate live from dead cells. The fluorescence shift caused by AE2P binding to the primary mouse T cells demonstrates binding to murine CD28 on the surface of these cells, while the lack of fluorescence shift observed with TGN1412 demonstrates a lack of binding of TGN1412 to primary mouse T cells.

### 5.4 Binding to Human CTLA-4

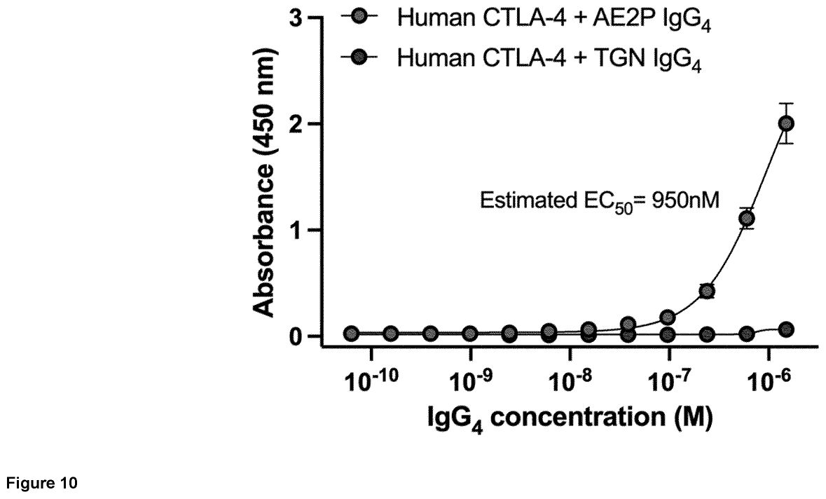

To test whether the AE2P and TGN1412 antibodies could also bind to CTLA-4, titration ELISA was performed by coating human CTLA-4 protein containing His6 Tag (SinoBiological, Cat: 11159-H08H) on NiNTA coated plates. After blocking with 2% milk-PBS, various concentrations of FITC-conjugate AE2P IgG₄ or TGN1412 IgG4 were added, and the signal was detected using rabbit anti-FITC followed by goat anti-rabbit HRP. Data was plotted at the different concentrations for binding to human CTLA-4 (FIG. 10). The results show that AE2P IgG₄, but not TGN1412 IgG₄, binds human CTLA-4.

### 5.5 In Vitro Super-Agonism Assay on Mouse T Cells

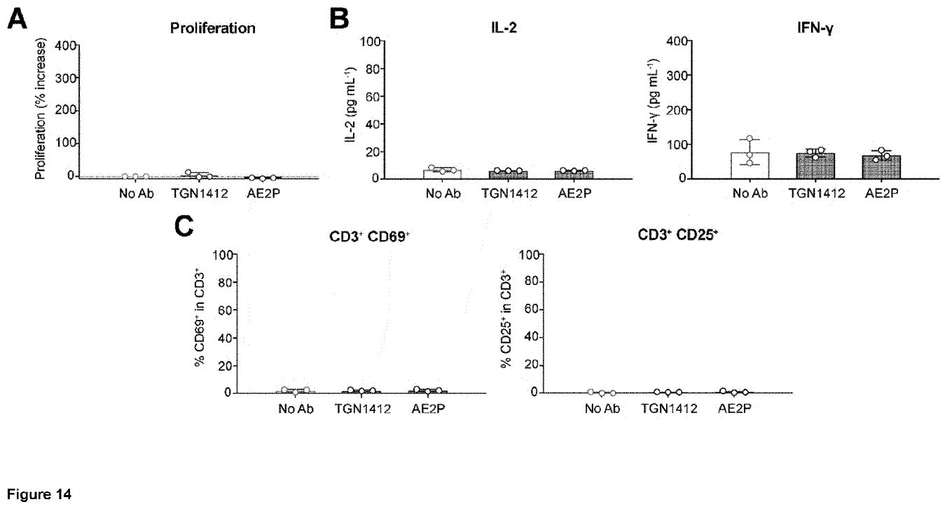

To test whether the AE2P and TGN1412 antibodies have super-agonistic activity in the mouse setting, the degree of mouse T cell proliferation induced by each antibody in the absence of CD3 stimulation was compared. Specifically, the AE2P antibody in IgG₄ format, and the TGN1412 antibody in IgG₄ format (used here as an isotype negative control, as it is not cross-reactive with murine CD28) were coated on a 6 well plate (10 μg per well). Primary mouse T cells (0.5 million per 1 mL in 3 mL) were added to the 6 well plate (in triplicate per experimental condition). Primary mouse T cells without added antibodies were also included as a control. The plate was incubated for 3 or 5 days at 37° C. in cell culture incubator. The cell proliferation assay was otherwise performed as mentioned above (section 3.1) after 5 days (FIG. 14A). These results show that AE2P IgG₄ did not induce proliferation of mouse T cells in the absence of CD3 stimulation, demonstrating that AE2P does not have super-agonist activity in the mouse setting.

FIG. 14B shows the quantification of cytokines (IL-2 and IFN-γ) by ELISA after 3 days, which was performed as set out in section 4.2, apart from the differences mentioned above. These results show that AE2P IgG₄ did not induce secretion of either IL-2 or IFN-γ by mouse T cells, confirming that this antibody does not have super-agonist activity in the mouse setting.

FIG. 14C shows the assessment of activation markers CD69 and CD25 by flow cytometry after 3 days, which was performed as set out in section 4.3, apart from the differences set out above. These results show that AE2P IgG₄ did not induce upregulation of the activation markers CD69 and CD25 in mouse T cells, confirming that this antibody does not have super-agonist activity in the mouse setting.

In summary, no mouse T-cell activation by the AE2P IgG₄ antibody was observed in the cell proliferation assay, the cytokine quantification assay, or the activation marker assay. Together, these results confirm this antibody does not have super-agonist activity in the mouse setting.

### 5.6 In Vitro Co-Stimulation Assay in Combination With the Anti-Murine CD3 Monoclonal Antibody 2C11 on Mouse T Cells

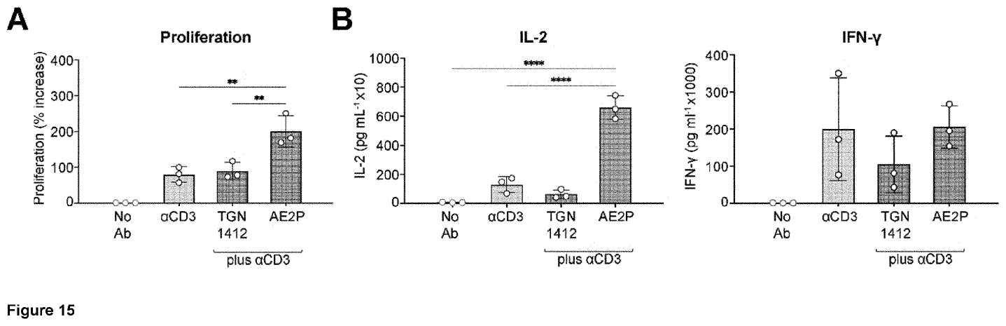

The co-stimulatory effect of AE2P and TGN1412 antibodies was tested by comparing the degree of mouse T cell proliferation induced by each antibody in the presence of CD3 stimulation. Specifically, 2C11, an activating monoclonal antibody against murine CD3 receptor (clone: 145-2C11, Biolegend, 100340), was coated on a 6 well plate (5 μg per 1 mL in 2 mL) for 2 hours at 37° C. in a cell culture incubator. Primary mouse T cells (0.5 million per 1 mL in 3 mL) were added to the 6 well plate (in triplicate per experimental condition). AE2P IgG₄ and TGN1412 IgG₄ (here used as an isotype negative control) were added at a concentration of 5 μg per 1 mL (total 15 μg) to each experimental condition. Primary mouse T cells without added antibodies were also included as a control. The plate was incubated for 3 days at 37° C. in a cell culture incubator. For cell proliferation, 200 μl from each triplicate was added to a 96 well plate, 20 μl of CellTiter 96 Aqueous One Solution Cell Proliferation assay was added, and the plate was incubated for 2 hours at 37° C. in a cell culture incubator. Absorbance at 490 nm was measured and percentage increase in cell proliferation (normalized to control condition) was plotted (FIG. 15A). These results demonstrate that only the AE2P IgG₄ antibody, and not the TGN1411 IgG₄ antibody, generated a co-stimulatory effect on the proliferation of mouse T cells in combination with an anti-CD3 antibody (2C11).

The co-stimulatory effect of AE2P and TGN1412 antibodies in the mouse setting was further tested by comparing the degree of cytokine release from mouse T cells induced by each antibody in the presence of CD3 stimulation, which was otherwise performed as mentioned above (Section 3.2). The results show that AE2P IgG₄, but not TGN1412 IgG₄, induced a significant increase in IL-2 production in mouse T-cells when combined with the anti-murine CD3 antibody 2C11 (FIG. 15B, left panel). No change was observed in IFN-γ release for either AE2P IgG₄ or TGN1412 IgG₄ (FIG. 15B, right panel).

### EXAMPLE 6—Epitope Mapping of AE2P

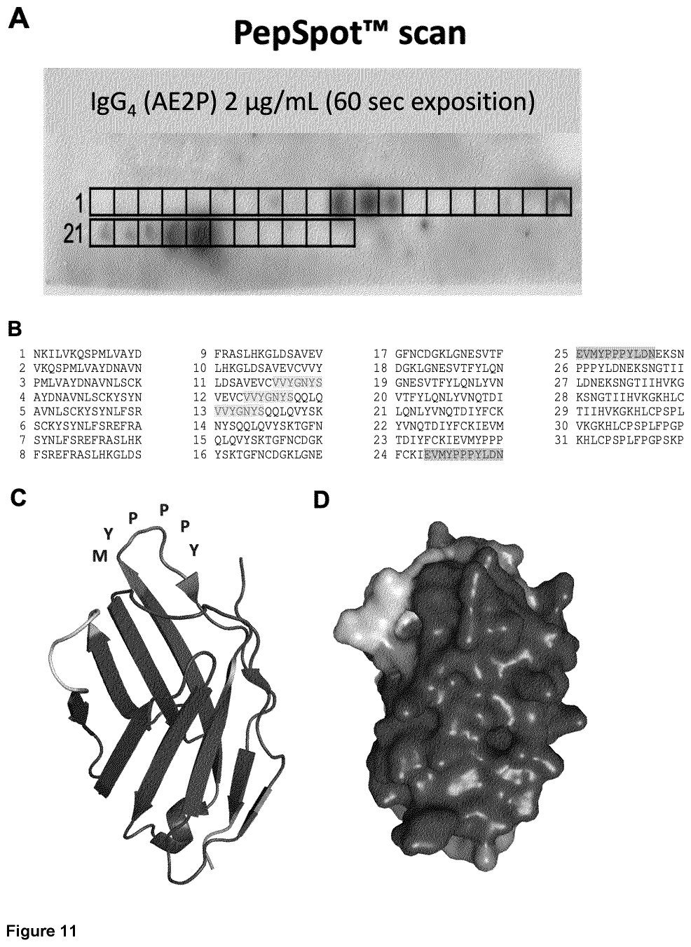

Epitope mapping was performed using synthetic peptides prepared by SPOT-synthesis scanning CD28 ECD sequence (FIG. 11B). The experiment was conducted by adding 2 μg/mL of AE2P IgG₄. The membrane was scanned to show important binding regions (spots) for AE2P (FIG. 11A). Potential binding residues were overlayed with the CD28 published crystal structure (PDB: 1YJD) (Evans et al., Nat Immunol, 2005, 6(3): 271-9) and a cartoon representation (FIG. 11C) and surface representation (FIG. 11D) are shown. These results show the epitope of CD28 recognized by the AE2P antibody, and the location of this epitope on the CD28 crystal structure.

### EXAMPLE 7—In Vitro Killing Assay With an AE2P Bispecific

To test the efficacy of the AE2P antibody in a bispecific format, AE2P was cloned in a bispecific T-cell engager (BiTE™) antibody format together with the antigen-binding site of the tumor-targeting anti-FAP antibody “7NP2” (WO2022/223824) and tested in combination with another BiTE™ molecule comprising the antigen-binding sites of the tumor-targeting anti-EDB antibody “L19” (U.S. Pat. No. 8,097,254) and the anti-CD3 antibody OKT3.

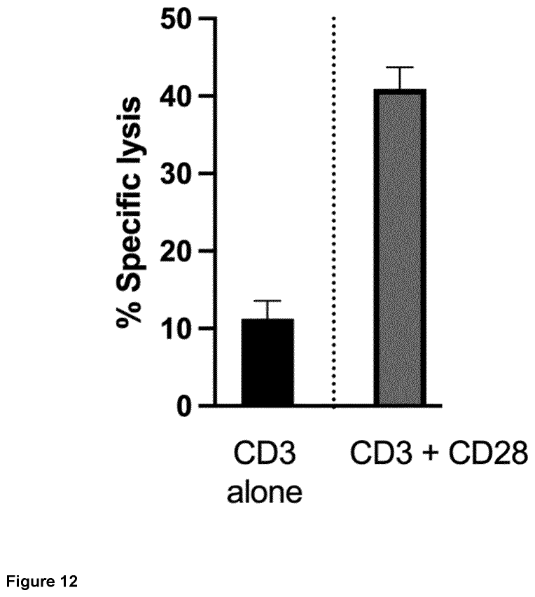

The WI-38 cell line, which expresses EDB, FAP and GFP, was used in an in vitro specific cell lysis assay using primary human T cells. 15,000 WI-38 cells were coated on a 96 well plate, and 75,000 primary human T cells were added. In triplicates, the “L19-OKT3” BiTE™ was added alone at 0.1 nM concentration or in combination with 10 nM of the “7NP2-AE2P” BiTE™. After 48 hours, all T cells and target cells were collected, washed with PBS then incubated for 30 minutes with Zombie NIR to discriminate between live and dead cells. Cells were washed once with FACS buffer and collected for flow cytometric analysis. WI-38 cells were gated based on GFP and the percentage of specific cell lysis was calculated. (FIG. 12). These results show that the combination of the 7NP2-AE2P BiTE™ with a CD3 binding BiTE™ (L19-OKT3) resulted in increased specific cell lysis of the WI-38 cell line when compared with L19-OKT3 alone. This indicates a potent and specific synergism between AE2P in a bispecific format and CD3-engaging bispecific antibodies.

### EXAMPLE 8—Different Formats for AE2P

AE2P is expected to find wide-ranging of application in cancer immunotherapy (i.e. as a booster in immunotherapy due to CD28 agonism and might act as a checkpoint inhibitor due to CTLA-4 binding), as well as application in the treatment of chronic infectious diseases due to its capacity to stimulate the immune system. FIG. 13 shows some potential mono-, bi-or multi-specific formats of AE2P, with or without conjugation to a binding moiety specific for a tumor target.

### EXAMPLE 9—Cloning, Characterization, and Activity of the Anti-CD3/Anti-EDB in BiTE™ Format

### 9.1 Cloning and Characterization of an Anti-CD3/Anti-EDB Bispecific Antibody

The anti-CD3/anti-EDB BiTE™ (clone PUB4 for the anti-CD3 (described in Liu Y, et al. (2022)) and clone L19 for the anti-EDB) was cloned in a mammalian expression vector pcDNA3.1+ with N-terminal ×6 His Tag. The anti-CD3/anti-EDB BiTE™ was purified by protein A affinity chromatography. The purified BiTE™ was analyzed by SDS-PAGE and by size exclusion chromatography using a Superdex 75 increase (S75i) column on an ÄKTA FPLC. The binding of the anti-CD3/anti-EDB BiTE™ was validated by flow cytometry against target cells (WI-38) and effector cells (purified human T cells).

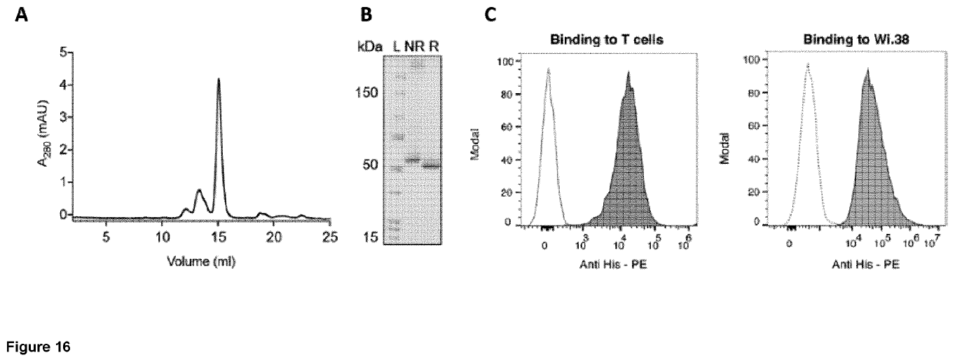

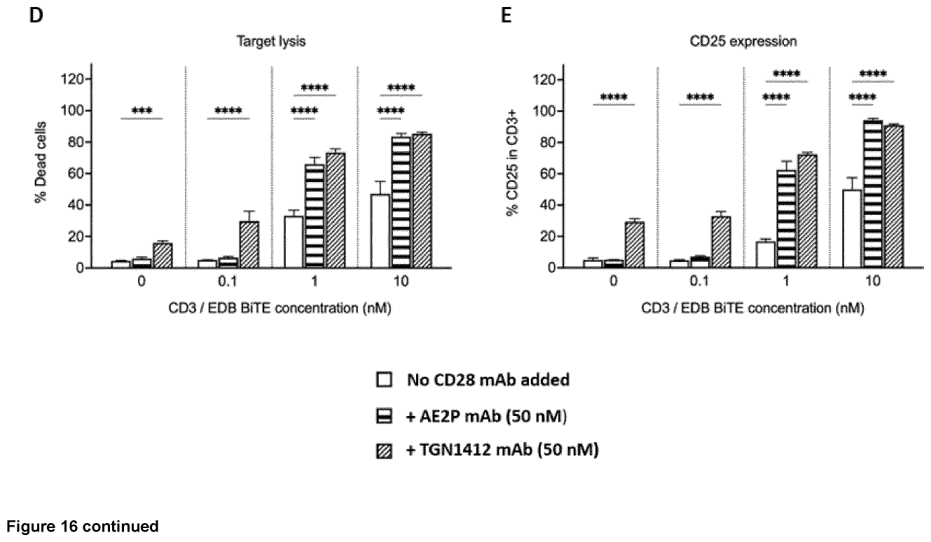

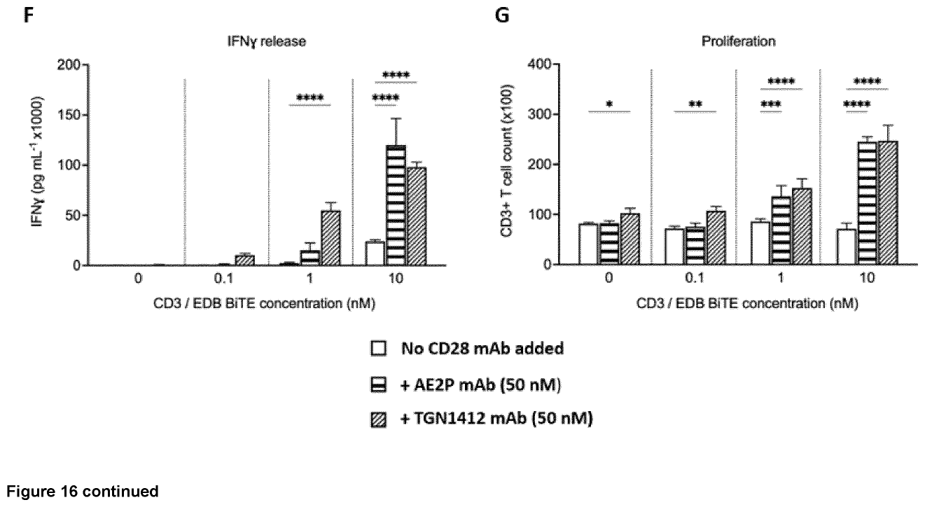

The sequence of the anti-CD3/anti-EDB BiTE™ molecule is shown in SEQ ID NO: 60. FIG. 16A shows the results of size exclusion chromatogram of the anti-CD3/anti-ED-B BiTE™. The anti-CD3/anti-ED-B BiTE™ was eluted from column as expected. FIG. 16B shows the results of SDS-PAGE analysis of the anti-CD3/anti-ED-B BiTE™. The anti-CD3/anti-ED-B BiTE™ had the expected size under non-reducing (NR) and reducing (R) conditions, respectively. FIG. 16C shows the binding of the anti-CD3/anti-ED-B BiTE™ to the effector human T-cells (left panel) and on the target human cells WI-38 (right panel) as analysed by flow cytometry. The grey area corresponds to the fluorescence intensity from anti-CD3/anti-EDB BiTE™ and the line to the fluorescence intensity from an isotype control.

### 9.2 In Vitro Co-Stimulation and Cell Killing Assay in Combination With an Anti-CD3/Anti-EDB Bispecific Antibody

The co-stimulatory effect of AE2P and TGN1412 antibodies on human PBMCs was tested by comparing the degree of T cell proliferation and target cell killing induced by each antibody in the presence of CD3 stimulation provided by an anti-CD3/anti-EDB BiTE™.

Specifically, WI-38 cells expressing the extracellular domain B (EDB) of fibronectin and eGFP were coated on a 96-well plate (20′000 cells/well). Freshly frozen human PBMCs (effector cells) were added to obtain an effector-to-target ratio of 5 to 1. Different concentrations of anti-CD3/anti-EDB BiTE™ were added in a 10-fold serial dilution (10, 1, and 0.1 nM). A condition of no added BiTE™ was also included as a negative control. AE2P IgG₄ and TGN1412 IgG₄ at a fixed concentration of 50 nM were added to each condition. After 4 days, cells were detached using an Accutase™ Cell detachment solution. The supernatant was collected to measure the level of IFN-γ release by ELISA. The remaining pellet and debris were washed with PBS before staining with Zombie Violet live/dead staining and incubated for 30 minutes at 4° C. in the dark. Cells were washed once with FACS buffer and stained with an antibody master mix containing anti-human CD3 APC and anti-human CD25 Alexa Fluor™ 647. Target cells were discriminated from effector cells via their eGFP expression, and the percentage of dead cells was calculated by gating on dead cells. The absolute count of live CD3⁺ T-cells was calculated to assess the proliferation. The percentage of CD25 expression on CD3⁺ T-cells was also assessed.

The results demonstrate that the anti-CD3/anti-EDB BiTE™ alone induced moderate target cell lysis at concentrations of 1 and 10 nM (33% and 47% of dead cells, respectively), but no effect was observed at concentrations of 0 and 0.1 nM (FIG. 16D). The combination of AE2P IgG₄ with an anti-CD3/anti-EDB BiTE™ resulted in approximately double the percentage of target cell lysis relative to the negative control. However, unlike TGN1412 IgG₄, which increased target cell lysis relative to the negative control at all anti-CD3/anti-EDB BiTE™ concentrations, for AE2P IgG₄ this synergistic effect was only observed at anti-CD3/anti-EDB BiTE™ concentrations at which the BiTE™ was active when added as a single agent (i.e., 1 and 10 nM). This demonstrates that, unlike TGN1412 IgG₄, AE2P IgG₄ only shows activity in the presence of CD3 stimulation. Similar results were also obtained when assessing CD25 expression (FIG. 16E), IFN-γ release (FIG. 16F), and T-cell proliferation (FIG. 16G). Statistical analysis of the data was performed by one-way ANOVA followed by Tukey's multiple comparison test (B&C) or by two-way ANOVA followed by Dunnett's multiple comparison test (E-H). *p<0.05; **p<0.01;***p<0.001; ****p<0.0001.

### EXAMPLE 10—Cloning and Characterization of the Anti-Human CEA IgG/anti-CD28 (scFv)₂ Bispecific Antibody (Format 1, FIG. 13)

The gene of the anti-human CEA IgG/AE2P (scFv)₂ bispecific antibody in IgG-(scFv)₂ (2+2) format (format 1, FIG. 13) was cloned, expressed using transient gene expression (TGE) in CHO-S cells, purified by protein A affinity chromatography, dialyzed, and stored in PBS. Bispecific antibodies were then characterized by size exclusion chromatography using an S200i 10/300 GL column on an ÄKTA FPLC. SDS-PAGE analysis was also performed with 4-12% Bis-Tris gel under reducing and non-reducing conditions. The sequences of the heavy chain and the light chain of the anti-human CEA IgG/AE2P (scFv)₂ bispecific antibody (2+2, format 1, FIG. 13) are shown in SEQ ID NOs 61 and 62 respectively.

To evaluate the binding of the anti-human CEA IgG/AE2P (scFv)₂ bispecific antibody, flow cytometry analysis on primary mouse T cells and target mouse C51 colon carcinoma cells expressing human CEA (C51.CEA) was performed by staining with 50 nM of bispecific antibodies. Cells were first blocked for unspecific binding for 30 minutes in FACS buffer (PBS, 2% FBS and 2 mM EDTA). After incubating for 1 hour with anti-human CEA IgG/AE2P (scFv)₂ bispecific antibody, cells were washed twice with FACS buffer (PBS, 2% FBS and 2 mM EDTA) and PE-conjugated anti-human secondary antibodies were added to detect the fluorescence shift in case of binding. Zombie near infrared (NIR) was used to discriminate live from dead cells.

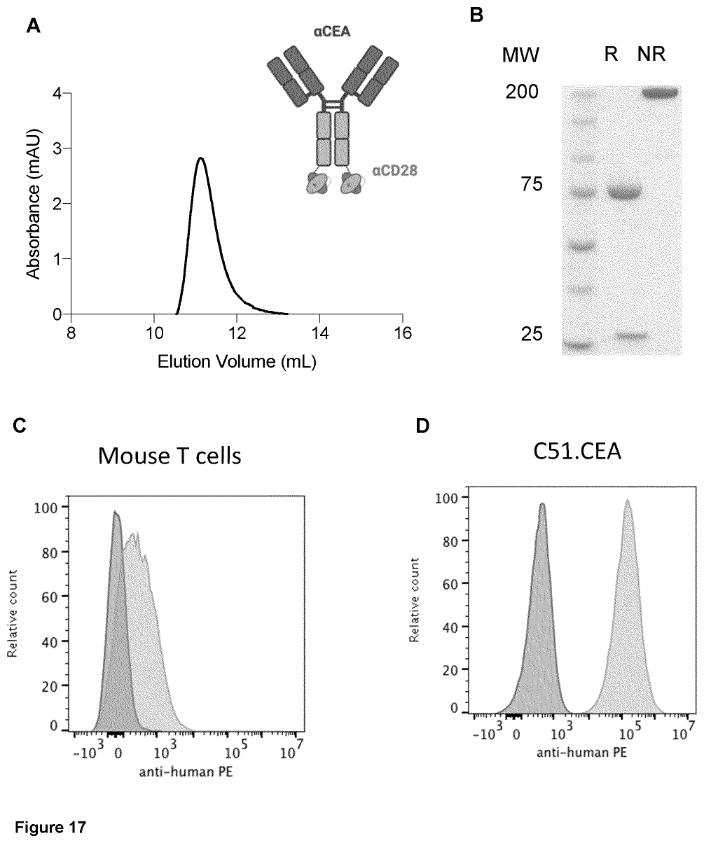

The anti-human CEA IgG/AE2P (scFv)₂ bispecific antibody showed good purity as evidenced by the single peak observed by SEC and the expected molecular weight under reducing and non-reducing conditions when analysed by SDS-PAGE (FIG. 17A and FIG. 17B). Binding of the bispecific antibody to both primary mouse T cells and target cells expressing human CEA (C51.CEA) was confirmed by flow cytometry (FIG. 17C and FIG. 17D).

### EXAMPLE 11—Cloning and Characterization of the Anti-CD28 IgG/Anti-human CEA (scFv)₂ Bispecific Antibody (format 2, FIG. 13)

The gene of the AE2P IgG/anti-human CEA (scFv)₂ bispecific antibody in IgG-(scFv)₂ (2+2) format, wherein the IgG is in IgG₄ format (format 2, FIG. 13) was cloned, expressed using transient gene expression (TGE) in CHO-S cells, purified by protein A affinity chromatography, dialyzed, and stored in PBS. Bispecific antibodies were then characterized by size exclusion chromatography using an S200i 10/300 GL column on an ÄKTA FPLC. SDS-PAGE analysis was also performed with 4-12% Bis-Tris gel under reducing and non-reducing conditions. The sequences of the heavy chain and the light chain of AE2P IgG/anti-human CEA (scFv)₂ bispecific antibody (2+2, format 2, FIG. 13) are shown in SEQ ID NOs 63 and 64 respectively.

To evaluate the binding of the AE2P IgG/anti-human CEA (scFv)₂ bispecific antibody, flow cytometry analysis on primary mouse T cells and target mouse C51 colon carcinoma cells expressing human CEA (C51.CEA) was performed by staining with 50 nM of bispecific antibodies. Cells were first blocked for unspecific binding for 30 minutes in FACS buffer (PBS, 2% FBS and 2 mM EDTA). After incubating for 1 hour with AE2P IgG/anti-human CEA (scFv)₂ bispecific antibody, cells were washed twice with FACS buffer and PE-conjugated anti-human secondary antibodies were added to detect the fluorescence shift in case of binding. Zombie near infrared (NIR) was used to discriminate live from dead cells.

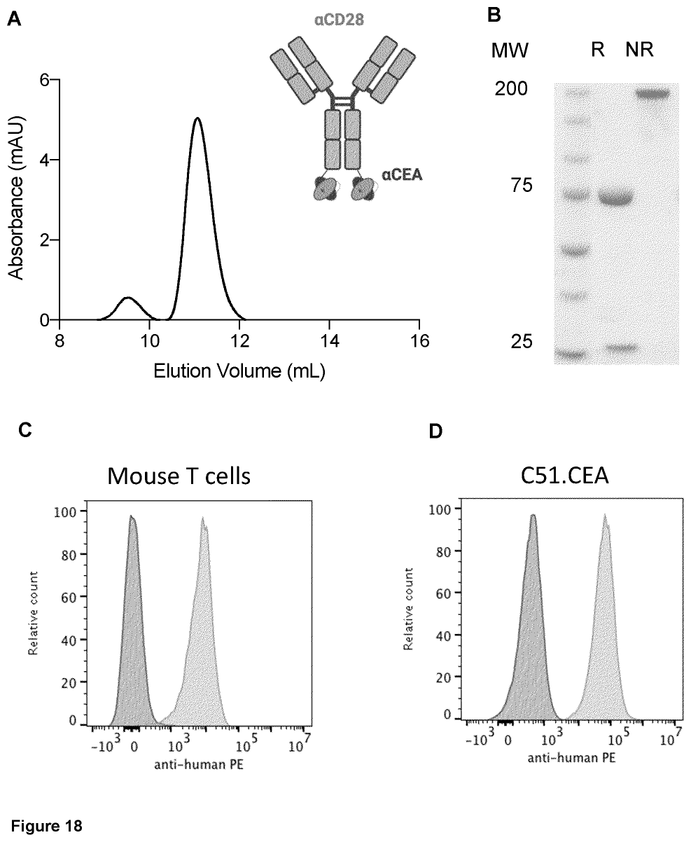

The AE2P IgG/anti-human CEA (scFv)₂ bispecific antibody showed good purity as evidenced by the single peak observed by SEC and the expected molecular weight under reducing and non-reducing conditions when analysed by SDS-PAGE (FIG. 18A and FIG. 18B). Binding of the bispecific antibody to both primary mouse T cells and target cells expressing human CEA (C51.CEA) was confirmed by flow cytometry (FIG. 18C and FIG. 18D).

### REFERENCES

A number of publications are cited above in order to more fully describe and disclose the invention and the state of the art to which the invention pertains. Full citations for these references are provided below. The entirety of each of these references is incorporated herein.
Allison, J. P., et al. J Immunol, 1982. 129(5): p. 2293-300.
Hedrick, S. M., et al. Nature, 1984. 308(5955): p. 149-53.
Yanagi, Y., et al. Nature, 1984. 308(5955): p. 145-9.
Davis, M. M. and P. J. Bjorkman, Nature, 1988. 334(6181): p. 395-402.
La Gruta, N. L., et al. Nat Rev Immunol, 2018. 18(7): p. 467-478.
Weiss, A. and J. D. Stobo. J Exp Med, 1984. 160(5): p. 1284-99.
Ohashi, P. S., et al. Nature, 1985. 316(6029): p. 606-9.
Weiss, A., et al. Annu Rev Immunol, 1986. 4: p. 593-619.
Janeway, C. A., Jr., Nature, 1988. 335(6187): p. 208-10.
Mueller, D. L., et al. Annu Rev Immunol, 1989. 7: p. 445-80.
Jenkins, M. K., et al. J Immunol, 1991. 147(8): p. 2461-6.
Bretscher, P. and M. Cohn. Science, 1970. 169(3950): p. 1042-9.
Damle, N. K., et al. J Immunol, 1983. 131(5): p. 2296-300.
June, C. H., et al. Mol Cell Biol, 1987. 7(12): p. 4472-81.
Martin, P. J., et al. J Immunol, 1986. 136(9): p. 3282-7.
Weiss, A., et al. J Immunol, 1986. 137(3): p. 819-25.
Freeman, G. J., et al. J Immunol, 1989. 143(8): p. 2714-22.
Freeman, G. J., et al. J Exp Med, 1993(a). 178 (6): p. 2185-92.
Freeman, G. J., et al. Science, 1993(b). 262 (5135): p. 909-11.
Azuma, M., et al. Nature, 1993. 366(6450): p. 76-9.
Caux, C., et al. J Exp Med, 1994. 180(5): p. 1841-7.
Brunet, J. F., et al. Nature, 1987. 328(6127): p. 267-70.
Linsley, P. S., et al. J Exp Med, 1991. 174(3): p. 561-9.
Peach, R. J., et al. J Exp Med, 1994. 180(6): p. 2049-58.
Krummel, M. F. and J.P. Allison, J Exp Med, 1995. 182(2): p. 459-65.
Leach, D. R., et al. Science, 1996. 271(5256): p. 1734-6.
Kwon, E. D., et al. Proc Natl Acad Sci U S A, 1997. 94(15): p. 8099-103.
Tacke, M., et al. Eur J Immunol, 1997. 27(1): p. 239-47.
Lühder, F., et al. J Exp Med, 2003. 197(8): p. 955-66.
Rodríguez-Palmero, M., et al. Eur J Immunol, 1999. 29(12): p. 3914-24.
Lin, C .H. and T. Hünig. Eur J Immunol, 2003. 33(3): p. 626-38.
Beyersdorf, N., et al. J Exp Med, 2005. 202(3): p. 445-55.
Pallardy, M. and T. Hunig. Br J Pharmacol, 2010. 161(3): p. 509-11.
Hanke, T. Lancet, 2006. 368(9547): p. 1569-70; author reply 1570.
Suntharalingam, G., et al. N Engl J Med, 2006. 355(10): p. 1018-28.
Stebbings, R., et al. J Immunol, 2007. 179(5): p. 3325-31.
Römer, P. S., et al. Blood, 2011. 118(26): p. 6772-82.
Eastwood, D., et al. Br J Pharmacol, 2010. 161(3): p. 512-26.
Kontermann, R. mAbs, 2012. 4:2, p. 182-197.
Bird et al., 1988 Science, 242, 423-426.
Huston et al., 1988 PNAS USA, 85, 5879-5883.
Holliger et al., 1993, Proc. Natl. Acad. Sci. USA 90 6444-6448.
Konterman and Muller, 1999, FEBS Letters 454:90-91.
Reiter et al., 1996, Nature Biotech, 14, 1239-1245.
Li et al., 1997, Protein Engineering, 10:731-736.
Liu et al., (2022) MABs Vol. 14, NO. 1, e2073632 (13 pages)
Batista et al., 1996, J. Exp. Med., 184:2197-205.
Crepeau and Ford, 2017, Expert Opin. Biol. Ther., 17, 1001-1012
Evans et al., 2005, Nat Immunol, 6(3): 271-9
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1-15. (canceled)

16. An antibody molecule that binds human CD28 and human CTLA-4, wherein the antibody molecule comprises a VH domain comprising a set of complementarity determining regions HCDR1, HCDR2 and HCDR3, and a VL domain comprising a set of complementarity determining regions LCDR1, LCDR2 and LCDR3, wherein:
the HCDR1, HCDR2 and HCDR3 comprise the amino acid sequences set forth in SEQ ID NOs 3, 4 and 5, respectively, and the LCDR1, LCDR2 and LCDR3 comprise the amino acid sequences set forth in SEQ ID NOs 6, 7 and 8, respectively.

17. An antibody molecule according to claim 16, wherein the VH domain comprises the amino acid sequence set forth in SEQ ID NO: 9 and/or the VL domain comprises the amino acid sequence set forth in SEQ ID NO: 10.

18. The antibody molecule according to claim 16, wherein the antibody molecule is human or humanised.

19. The antibody molecule according to claim 16, wherein the antibody molecule comprises or consists of: a single chain Fv (scFv), a diabody (Db), a single-chain diabody (scDb), a small immunoprotein (SIP), an IgG1 molecule, an IgG2a molecule, or an IgG4 molecule.

20. An antibody molecule according to claim 19, wherein the antibody molecule comprises the amino acid sequence set forth in:
SEQ ID NO: 11;
SEQ ID NO: 15;
SEQ ID NO: 19;
SEQ ID NO: 65; or
SEQ ID NO: 25;
or wherein the antibody molecule comprises the heavy chain amino acid sequence set forth in SEQ ID NO: 22, SEQ ID NO: 17, or SEQ ID NO: 13, and the light chain amino acid sequence set forth in SEQ ID NO: 14.

21. A conjugate comprising an antibody molecule according to claim 16 and a pro-inflammatory agent or a radioisotope.

22. An antibody molecule according to claim 16, wherein the antibody further comprises a second antigen-binding site which binds a tumor associated antigen, optionally wherein the tumor associated antigen is selected from the group consisting of: fibroblast activation protein (FAP), the ED-A, ED-B or IIICS isoform of fibronectin, CAIX, CEA, Mucin-16, PSMA, or the A, A1, A2, B, C or D isoform of tenascin C.

23. An antibody molecule according to claim 22, wherein the antibody molecule is:
(i) a bispecific T-cell engager;
(ii) a scDb-scFv;
(iii) an IgG-(scFv); or
(iv) an IgG-(scFv)₂.

24. The antibody molecule according to claim 23, wherein:
the antibody molecule is a bispecific T-cell engager and comprises the amino acid sequence set forth in SEQ ID NO: 23;
the antibody molecule is an IgG-(scFv)₂ and comprises the heavy chain amino acid sequence set forth in SEQ ID NO: 61 and the light chain amino acid sequence set forth in SEQ ID NO: 62; or
the antibody molecule is an IgG-(scFv)₂ and comprises the heavy chain amino acid sequence set forth in SEQ ID NO: 63 and the light chain amino acid sequence set forth in SEQ ID NO: 64.

25. The antibody molecule or conjugate according to claim 16 for use in a method of treatment of the human or animal body by therapy.

26. The antibody molecule or conjugate according to claim 16 for use in a method of treating cancer in a patient.

27. The antibody molecule for use according to claim 25, wherein the method further comprises administering a second therapeutic agent to the patient, wherein the second therapeutic agent is selected from:
(i) chemotherapy,
(ii) radiation therapy;
(iii) a checkpoint inhibitor;
(iv) an agonist which binds GITR, 0X40, or 4-1 BB;
(v) a costimulatory anti-CD28 bispecific antibody; or
(vi) an antibody which binds human CD3.

28. A nucleic acid molecule or expression vector encoding an antibody molecule or conjugate according to claim 16 or a host cell comprising said nucleic acid or expression vector.

29. A method of producing an antibody molecule or conjugate, the method comprising culturing the host cell of claim 28 under conditions for expression of the antibody molecule or conjugate, and optionally further isolating and/or purifying the antibody molecule or conjugate following expression.
